(19) United States
(12) Patent Application Publication
(10) Pub. No.: US 2004/0191926 A1
(43) Pub. Date: Sep. 30, 2004

(54) Ptp1b inhibitors and ligands

(76) Inventors: Zhong-Yin Zhang, Scarsdale, NY (US); David S Lawrence, Scardale, NY (US)

Correspondence Address: AMSTER, ROTHSTEIN & EBENSTEIN, 90 PARK AVENUE, NEW YORK, NY, 10016

(21) Appl. No.: 10/490,836

(22) PCT Filed: Sep. 25, 2002
(86) PCT No.: PCT/US02/30492

(30) Foreign Application Priority Data
Sep. 26, 2001 (US) 60325009

(51) Int. Cl. C07F 9/02; A61K 31/66; G01N 33/543

(52) U.S. Cl. 436/518; 514/109; 558/73

(57) ABSTRACT

Methods for discovery of enzyme ligands and inhibitors are disclosed. The methods comprise the creation and testing of combinatorial libraries comprising an active site-targeted component, a linker component and a peripheral site-targeted component. The methods also comprise a novel assay for determining whether a compound is a ligand of an enzyme. The assay evaluates whether the compound can inhibit the binding of a known ligand of the active site of the enzyme to a mutant of the enzyme that can bind the enzyme substrate but cannot catalyze an enzymatic reaction with the substrate. Various ligands and inhibitors of protein tyrosine phosphatase 1B (PTP1B) are also disclosed. These ligands and inhibitors were discovered using the above methods. One particular inhibitor discovered using the invention methods has the highest specificity and affinity of any PTP1B inhibitor discovered to date.

### STATEMENT REGARDING FEDERALLY SPONSORED RESEARCH

This invention was made with U.S. Government support under National Institutes of Health Grant No. GMS5242. The Government has certain rights to the invention.

### BACKGROUND OF THE INVENTION

(1) Field of the Invention.

The present invention relates to ligands and inhibitors of enzymes. More specifically, the present invention relates to methods for discovering and evaluating ligands and inhibitors for an enzyme, and specific inhibitors of protein tyrosine phosphatase 1B, which were found using the above methods. Additionally, the invention relates to methods of using those inhibitors for therapy against obesity and type II diabetes.

(2) Description of Related Art.

Enzyme inhibitors are known for a vast number of enzymes. They are useful for therapeutic applications as well as for research purposes (see, e.g., refs. 42-44). An important group of enzymes where improved enzyme inhibitors would be useful are protein tyrosine phosphatases.

The initiation, propagation, and termination of signaling events controlling many cellular processes are determined by the level of tyrosine phosphorylation. Phosphotyrosine level, in turn, is maintained in an exquisite balance by the reciprocal activities of protein-tyrosine kinases and protein-tyrosine phosphatases (PTPases). To date, a large number of PTPases has been identified. Because balanced protein tyrosine phosphorylation is critical for the maintenance of cellular homeostasis, it is not surprising that PTPase malfunction has been linked to many human diseases (1). Consequently, in those instances where PTPase activity is inappropriately high, PTPase inhibitors may provide a valuable new family of therapeutic agents. However, drug development targeted to PTPases was not seriously considered until recently. A major concern is that a PTPase may regulate multiple signaling pathways, while at the same time a single pathway may be controlled by several PTPases. Thus, PTPase inhibition was thought to likely give rise to unwanted side effects. Significant progress has been made that is beginning to alleviate this concern.

PTP1B has been shown to be a negative regulator of insulin (2-4) and leptin (45, 46) signaling. PTP1B^{-/-} mice display increased insulin receptor and insulin receptor substrate-1 phosphorylation and enhanced sensitivity to insulin in skeletal muscle and liver (5, 6). In addition, PTP1B^{-/-} mice have remarkably low adiposity and are protected from diet-induced obesity. Perhaps most importantly, these mice appeared to be normal and healthy, indicating that regulation of insulin signaling by PTP1B is tissue and cell type specific. These observations suggest that specific PTP1B inhibitors might be free of side effects and highlight the potential of selective therapeutic efficacy in targeting PTP1B (anti-diabetes/obesity) even though PTP1B is expressed ubiquitously.

Clearly, however, potent and selective PTPase inhibitors are required before therapeutic intervention with PTPase inhibitors can become a reality. Thus, there is intense interest in obtaining specific and potent PTPase inhibitors for biological studies and pharmacological development.

Structural and mutational studies have shown that amino acids involved in catalysis or formation of the pTyr binding site (the active site) are conserved (7-9), indicating that PTPases utilize similar mechanisms for phosphomonoester hydrolysis and pTyr recognition. Can specificity be achieved by targeting the PTPase active site for inhibitor development? A similar question was raised in the protein kinase field due to the structural conservation of the ATP binding site. In spite of the latter, a number of highly selective, ATP-binding site-targeted, protein kinase inhibitors have been described (10, 11). In several instances, structural studies reveal that specificity comes from the fact that only a portion of each inhibitor interacts with the residues that bind ATP, whereas the rest of the molecule makes contact with residues situated outside the ATP-binding pocket (11).

### BRIEF SUMMARY OF THE INVENTION

The present invention is directed toward methods useful for discovery of ligands and inhibitors of enzymes, as well as compositions resulting from those methods comprising a combinatorial library for discovery of ligands and inhibitors of protein tyrosine phosphatase 1B (PTP1B). Various novel PTP1B ligands and inhibitors are also disclosed.

The methods of the present invention utilize a combinatorial approach that is designed to target both the active site and a unique peripheral site of enzymes, in particular PTP1B. Compounds that can simultaneously associate with both sites are expected to exhibit enhanced affinity and specificity. We also describe a novel affinity-based high-throughput assay procedure that can be used for PTPase inhibitor screening. The combinatorial library/high-throughput screen protocols furnished several small molecule PTP1B inhibitors, including one that is both potent (Kᵢ=2.4 nM) and selective (little or no activity against a panel of phosphatases including Yersinia PTPase, SHP1, SHP2, LAR, HePTP, PTPa, CD45, VHR, MKP3, Cdc25A, Stp1, and PP2C). These results demonstrate that it is possible to acquire potent, yet highly selective inhibitors for individual members of the large PTPase family of enzymes.

Accordingly, in some embodiments, the invention is directed to compounds comprising an active site-targeted component, a linker component, and a peripheral site-targeted component. In these embodiments, the linker component is covalently bound to the active site-targeted component and the peripheral site-targeted component is covalently bound to the linker component. Further, the active site-targeted component has the formula as in compound 3 of FIG. 1, and the linker component and the peripheral site-targeted component are any organic molecule of less than 500 Dalton

In other embodiments, the invention is directed to ligands of protein tyrosine phosphatase 1B (PTP1B) with an active site-targeted component, a linker component, and a peripheral site-targeted component, the ligand comprising the formula of compound 3 of FIG. 1. In these embodiments, the linker component and the peripheral site-targeted component are selected from the group consisting of the following elements of FIGS. 3 and 2, respectively: 4A, 4B, 4C, 4E, 4F, 5A, 5B, 5C, 5F, 6A, 6B, 6E, 6F, 6H, 7A, 7B, 7C, 7E, 7F, 7H, 8A, 8B, 8C, 8F, 8H, 9A, 9B, 9C, 9F, 9H, 10A, 10B, 10C, 10F, 10H, 11A, 11B, 11C, 11D, 11E, 11F, 11G, 11H, 12A, 12B, 12C, 12F, 12G, 12H, 13A, 13B, 13C, 13D, 13E, 13F, 13G, 13H, 14A, 14B, 14C, 15A, 15B, 15C, 15E, 15F, 15H, 16A, 16B, 16C, 16F, 16H, 17A, 17B, 17C, 17E, 17F, 17H, 18A, 18B, 18C, 18E, 18F, 18G, 18H, 19A, 19B, 19C, 19F, 20A, 20B, 20C, 20E, 10F, 20G, 20H, 21A, 21B, 21C, 21D, 21E, 21F, 21G, 21H, 22A, 22B, 22C, 22D, 22E, 22F, 22G, 23H, 24A, 24B, 24C, 24D, 24E, 24F, 24G, 24H, 25F, 26A, 26B, 26C, 26E, 26F, 26G, and 26H. Additionally, the ligands of these embodiments comprise at least one phosphate group.

The invention is also directed to inhibitors of protein tyrosine phosphatase 1B (PTP1B) with an active site-targeted component, a linker component, and a peripheral site-targeted component. In these embodiments, the inhibitor comprises any of the above ligands, wherein the any phosphate groups are substituted with a difluorophosphonate group.

Additionally, the invention is directed to compositions comprising any of the above inhibitors, in a pharmaceutically acceptable excipient.

In additional embodiments, the invention is directed to methods of preventing or treating obesity in a patient. The methods comprise administering to the patient one of the above compositions.

The invention is further directed to methods of preventing or treating Type II diabetes in a patient. These methods also comprise administering to the patient one of the above compositions.

The invention is also directed to methods of evaluating whether a compound is a ligand of an enzyme. The methods comprise the steps of (a) combining a known active site ligand of the enzyme with the compound and a mutant of the enzyme, wherein the mutant is capable of binding to a substrate of the enzyme, but not catalyzing the chemical conversion of the substrate; and (b) determining whether the compound is capable of competing for binding of the known ligand to the mutant of the enzyme, wherein the capacity of the compound to compete for binding indicates that the compound is a ligand for the enzyme.

Additionally, the invention is directed to combinatorial libraries for discovering a ligand of a protein tyrosine phosphatase. These libraries comprise more than one form of compound 3 of FIG. 1, wherein X and Y are each independently any organic molecule of less than 500 Dalton.

### BRIEF DESCRIPTION OF THE DRAWINGS

FIG. 1 is a compound for a combinatorial library, designated structure 3 or compound 3. The library is directed to the discovery of ligands and inhibitors of protein-tyrosine phosphatases.

FIG. 2 depicts terminal diversity elements, or peripheral site-targeted components, used in the library of the general structure 3 to target a unique peripheral site.

FIG. 3 depicts linkers used to connect the N-terminal diversity elements and pTyr. In the case of 26, the terminal elements are directly linked to pTyr.

FIG. 4 depicts Scheme I, utilized for the parallel synthesis of a library of compounds targeting both the active site and a unique adjacent site of PTP1B.

FIG. 5 depicts Scheme II, utilized for the synthesis of the hydrolytically resistant difluorophosphonate analog (32) of B.

FIG. 6 depicts Scheme III, utilized for the synthesis of the difluorophosphonate-containing unnatural amino acid 38.

FIG. 7 depicts the results from the ELISA-based screening of library members at 250 nM concentration. The potency of the library members for PTP1B is represented by the ability of the compounds to inhibit (expressed as percent inhibition) the binding of GST-PTP1B/C215S to the biotinylated DADEpYL-NH₂ peptide immobilized on avidin-coated microtiter plate wells.

FIG. 8 depicts the chemical structures of the reference compound 39 and the nonhydrolyzable analog of 21B, compound 40.

FIG. 9 depicts the chemical structures of compound 40 and its analogs 40A, 40B, and 40C.

FIG. 10 are confocal micrographs of CHO/HIRc cells treated with compound 40B, demonstrating that the compound enters the cells. Panel (A) is a fluorescent micrograph; Panel (B) is a light micrograph.

FIG. 11 shows a western blot evaluating binding of anti-phosphotyrosine antibodies to a blot of a PAGE gel of electrophoresed extracts of CHO/Hir cells, showing the effects of compound 40A and insulin on tyrosine phosphorylation of the insulin receptor (Irβ) and the insulin receptor substrate-1 (IRS-1).

FIG. 12 shows western blots evaluating binding of anti-phospho-AKT-1 (α-phospho-Akt1) and anti-Akt1 (α-Akt1) antibodies to a blot of a PAGE gel of electrophoresed extracts of CHO/Hir cells, showing the effect of compound 40A and insulin treatment on Akt phosphorylation in CHO/Hir cells.

FIG. 13 shows western blots evaluating binding of anti-phospho-ERK (α-phospho ERK 44/42) and anti-ERK (α-ERK) antibodies to a blot of a PAGE gel of electrophoresed extracts of CHO/Hir cells, showing the effect of compound 40A and insulin treatment on MAPK phosphorylation in CHO/Hir cells.

FIG. 14 is a bar graph showing increased glucose uptake in CHO/Hir cells treated with compound 40A.

FIG. 15 is a graph showing increased glucose uptake in L6 myotubes treated with compound 40A. Circles—untreated myotubes; Squares—myotubes treated with compound 40A at 125 nM.

### DETAILED DESCRIPTION OF THE INVENTION

Abbreviations: Ahx, 6-aminohexanoic acid; Boc, tert-butoxylcarbonyl; BOP, benzotriazole-1-yl-oxy-tris-(dimethylamino)-phosphonium hexafluorophosphate; DAST, (diethylamino)sulfur trifluoride; DIC, 1,3-diisopropylcarbodiimide; DIPEA, N,N-diisopropylethylamine; DMA, N,N-dimethylacetamide; DMAP, 4-(dimethylamino)pyridine; DMF, N,N-dimethylformamide; DMSO, dimethyl sulfoxide; DTT, dithiothreitol; EDT, 1,2-ethanedithiol; ELISA, enzyme-linked immunosorbent assay; ESI-MS, electron spray ionization-mass spectroscopy; Fmoc, 9-fluorenylmethoxycarbonyl; Fmoc-Osu, N-(9-fluorenylmethoxycarbonyloxy)succinimide; HBTU, 2-(1H-benzotriazol-1-yl)-1,1,3,3-tetramethyluronium hexafluorophosphate; HMPA, hexamethylphosphoramide; HPLC, high performance liquid chromatography; HOBt, N-hydroxybenzotriazole; LHMDS, lithium bis(trimethylsilyl)amide; MOLDI-TOF, matrix-assisted laser desorption ionization-time of flight; MS, mass spectroscopy; NMM, N-methylmorpholine; NMR, nuclear magnetic resonance; PTPase, protein tyrosine phosphatase; PTP1B, protein tyrosine phosphatase 1B; pTyr, O-phospho-L-tyrosine; PyBOP, benzotriazole-1-yl-oxy-tris-pyrrolidino-phosphonium hexafluorophosphate; TFA, trifluoroacetic acid; TFFH, tetramethylfluoroformamidinium hexafluorophosphate; THF, tetrahydrofuran; TIS, triisopropylsilane; TMSBr, bromotrimethylsilane; TMSI, iodotrimethylsilane; Tris, tris(hydroxymethyl)aminomethane; TSTU, O-(N-succinimidyl)-1,1,3,3-tetramethyluronium tetrafluoroborate.

The present invention is directed toward methods of discovering enzyme ligands and inhibitors, and the use of those methods in the discovery of several high affinity ligands and corresponding inhibitors of protein tyrosine phosphatase 1B (PTP1B) that are highly specific. The methods are based on the creation of a combinatorial library that targets the active site of the enzyme along with a peripheral site.

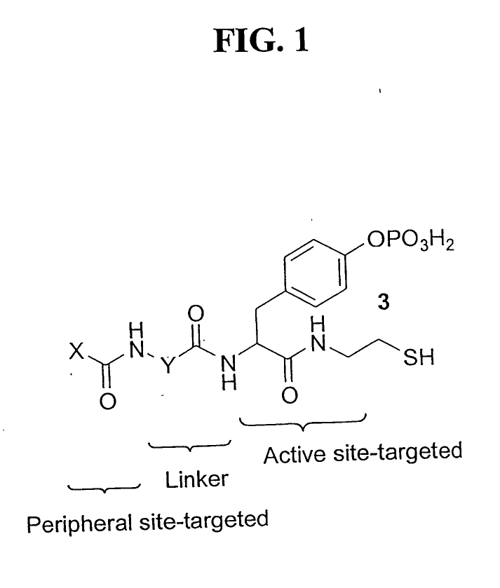

The combinatorial library utilized in the methods of the invention is directed toward the discovery of ligands of the enzyme. The library comprises compounds that have an active site-targeted component mimicking the active site of known substrates of the enzyme, a linker component linked to the active site, and a peripheral site-targeted component. See, e.g., compound 3, shown in FIG. 1, showing the active site, linker and peripheral site components of a PTP1B combinatorial library.

The active site-targeted component of the library members can be any appropriate compound that is known as a substrate for the particular enzyme under investigation. Such active site components are known for a plethora of enzymes and a suitable active site could be selected for any particular enzyme by a skilled artisan without undue experimentation. For any combinatorial library, more than one known active site-targeted component could be selected. However, in preferred embodiments, only one active site-targeted component is present in all of the members of the library. In the most preferred embodiments, this active site-targeted component is the active site target that is present in the known substrate of the enzyme that has the highest affinity for the enzyme. Having only one active site component for all library members is preferred because it would decrease the complexity of the library and allow the focus of the investigation to be directed to the linker and peripheral site components, where variations would be expected to impart widely varying enzyme affinity and specificity characteristics to the library members. For the exemplary PTPase enzymes, a preferred active site component is shown in FIG. 1, as pTyr in compound 3.

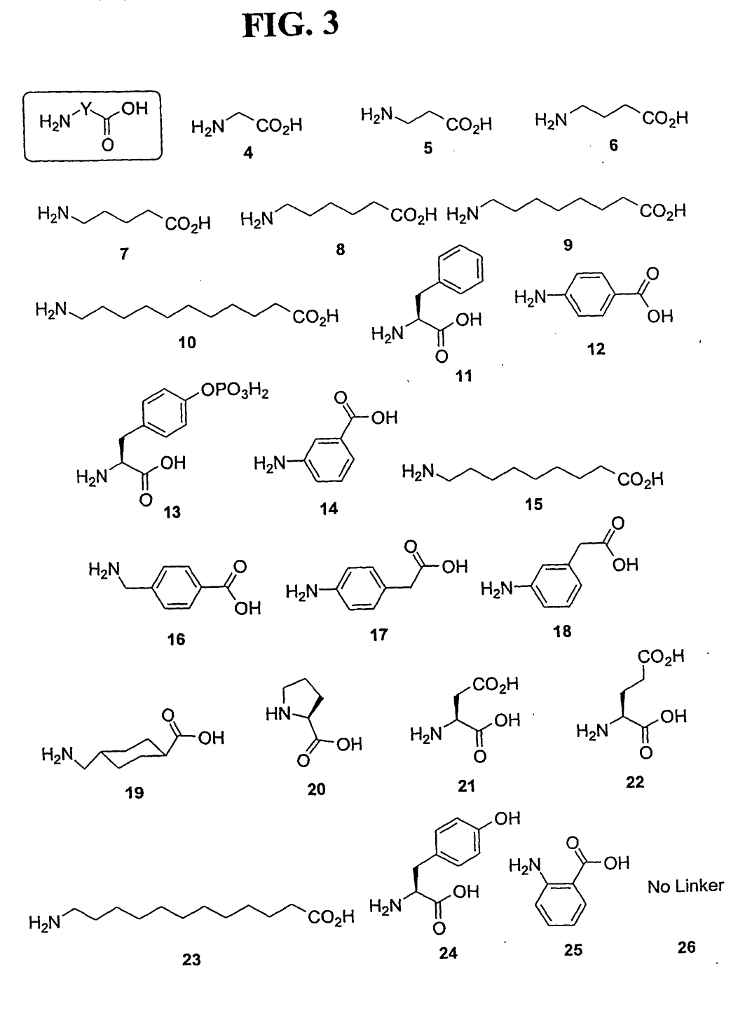

The linker component serves to provide a spacer and desirable charge characteristics between the active site and peripheral site components of the library members. As such, the linker is covalently bound to both the peripheral site-targeted and active site-targeted components, preferably by an amide bond, as in compound 3. An example of a useful set of linkers is shown in FIG. 3. As indicated in FIG. 3, a linker set as defined herein can include a null member, wherein the peripheral site component is directly covalently bound to the active site component. Preferably, the linker component is less than 500 Dalton. In other preferred embodiments, the linker component consists of carbon, oxygen, nitrogen, and/or hydrogen. However, the use of other atomic elements is also possible.

The peripheral site-targeted component of the library members serves to target areas near the active site to increase specificity and affinity of the enzyme ligand/inhibitor interaction. As used herein, “target” refers to the ability of the component, or the library members themselves, to reversibly bind to the enzyme active site or areas near the active site. As is well known in the art, such binding is enhanced by the presence of complementary shape and charge characteristics between the component/library member and enzyme active site.

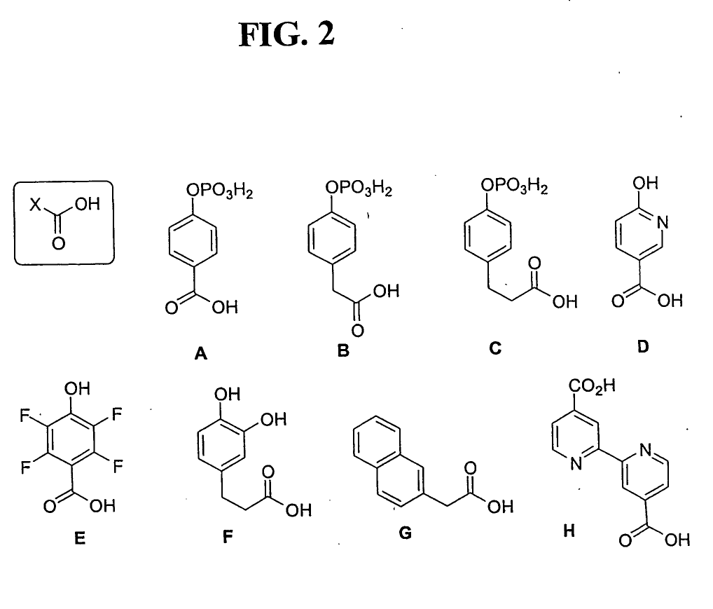

The peripheral site-targeted component preferably consists of carbon, oxygen, nitrogen, phosphorous and/or hydrogen. However, as with the linker component, the use of other atomic elements is also envisioned. The peripheral site component is also preferably less than about 500 Dalton. A useful set of peripheral site-targeted components is shown in FIG. 2.

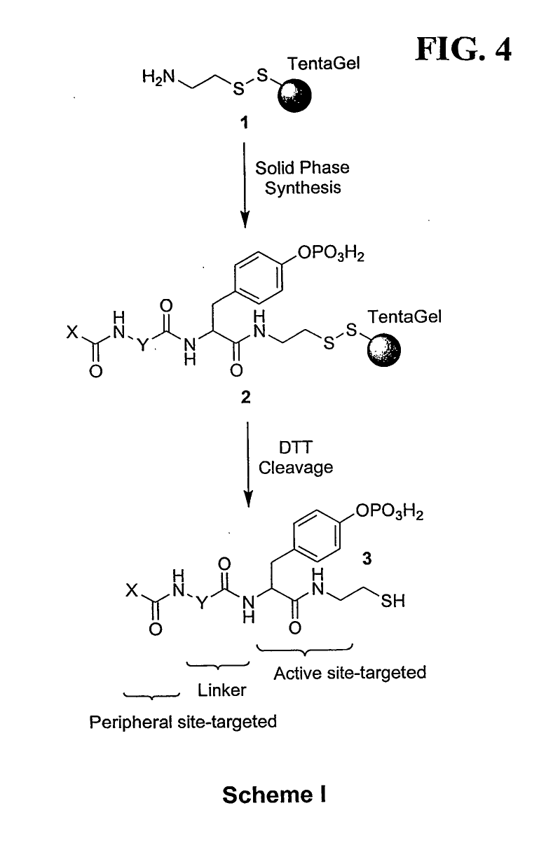

The synthesis of the various library members can be by any appropriate method known in the art. Preferably the library members are synthesized on a resin by known solid phase methods. An example is solid phase synthesis on a disulfide-modified Tentagel S NH₂ resin using Fmoc chemistry. See Example 1 and FIGS. 4-6 for exemplary methods used in the synthesis of various library members and inhibitor analogs used in the discovery of PTP1B ligands and inhibitors.

As envisioned herein, the compounds representing the various components of the library, or any other compound to be tested for ligand activity, are evaluated for activity as a ligand of the targeted enzyme by a novel assay method. The method comprises the following steps:

(a) combining a known active site ligand of the enzyme with the compound and a mutant of the enzyme, wherein the mutant is capable of binding to a substrate of the enzyme, but not catalyzing the chemical conversion of the substrate;

(b) determining whether the compound is capable of competing for binding of the known ligand to the mutant of the enzyme, wherein the capacity of the compound to compete for binding indicates that the compound is a ligand for the enzyme.

This assay is designed to detect ligands to the targeted enzyme by evaluating the ability of the candidate ligand to compete for the binding of a known active site ligand of the enzyme to the mutant of the enzyme. This competitive assay is preferred over simply an assay for enzyme activity or an assay that evaluates the ability of the candidate to bind to the enzyme because this competitive assay requires the candidate ligand to displace a known active site ligand of the enzyme. A ligand that is able to displace a known active site ligand of the enzyme must necessarily have sufficient affinity for the active site to be able to displace the known active site ligand from that site. Thus, the assay selects for high affinity active site ligands and not just compounds that are efficient substrates but not necessarily high-affinity ligands. The competitive assay is particularly useful for discovering compounds that inhibit the enzyme because superior inhibitors would be expected to have high affinity for the active site.

Since the assay method of the present invention is designed to measure ligand affinity and not the ability of a candidate ligand to serve as an enzyme substrate, the assay utilizes a mutant of the enzyme that retains active site ligand binding activity but exhibits no activity on a substrate. Such mutants are well known for many enzymes, and the utilization of this assay for determining ligand activity for any of those enzymes would not require undue experimentation. An example of a mutant enzyme useful for this assay method is the C215S mutant of PTP1B (33).

The competitive assay disclosed above preferably utilizes a solid phase to which one of the assay components is bound. The solid phase is not narrowly limited to any particular matrix, and the assay could be performed on beads, microtiter plates, paper, membranes, or any other such matrix, for example the matrix described in U.S. Pat. No. 6,225,131. Preferably, the matrix allows for high throughput screening of candidate ligands.

In the solid phase embodiments of the assay, the assay could be performed by first binding the mutant to the solid phase, then adding the known ligand and the candidate ligand. The ability of the candidate ligand to compete with the known ligand for binding to the mutant is then determined by any of a number of well-known methods, for example utilizing an antibody to the known ligand, or by using a known ligand that is tagged, e.g., with a radioactive or fluorescent label, or a hapten that can be quantified, such as biotin (which can be measured, e.g., using labeled avidin or avidin with an antiavidin antibody) or digoxygenin (which can be measured using an anti-digoxygenin antibody). The ability of the candidate ligand to compete for active site binding with the known ligand is determined by quantifying the known ligand bound to the solid phase and comparing the amount of such bound known ligand with the amount of known ligand that is bound without the candidate ligand.

In alternative solid phase embodiments, the known ligand is bound to the solid phase. The candidate ligand and the mutant are then added. In these embodiments, the ability of the candidate ligand to compete for active site binding with the known ligand is determined by quantifying the mutant bound to the solid phase and comparing the amount of such bound mutant with the amount of mutant that is bound without the candidate ligand. The bound mutant can be quantified by using a mutant labeled, e.g., with a radioactive or fluorescent label, with a hapten (that can be quantified with an anti-hapten antibody), or with an antibody to the mutant.

As used herein, the term “antibody” includes those of monoclonal or polyclonal origin, fragments that retain at least one binding site, or any other variant that would be recognized as equivalent in utility to a whole antibody. In the above methods, the skilled artisan would recognize that an antigen or hapten quantified by an antibody is quantified by quantifying the antibody bound to the antigen or hapten, for example by using a labeled antibody or a second labeled antibody that specifically binds to the antibody that binds to the antigen or hapten.

An illustration of the assay of the present invention is provided in Example 1. In that assay, a known ligand/substrate of PTPL1B, DADEpYL, is biotinylated and bound to an avidin-coated microtiter well. The candidate ligand is then added to the microtiter well along with a recombinant fusion protein of glutathione S transferase (GST) and the C215S mutant of PTP1B (GST-PTP1B/C215S). After incubation and washing, bound C215S is quantified by adding an anti-GST antibody, then a horseradish peroxidase-conjugated mouse anti-rabbit antibody. After washing, the bound peroxidase is quantified. That measurement is compared with the determination of bound GST-PTP1B/C215S when the candidate ligand is not added. A smaller value of bound peroxidase in the wells with the candidate ligand than in the wells without the candidate ligand indicates that the candidate ligand is a ligand of PTP1B.

The utilization of the above methods to identify ligands of the target enzyme allows the development of inhibitors of the enzyme. In many cases, the ligand itself can serve as an inhibitor, if the enzyme is unable to utilize the ligand as a substrate. Also, if the ligand is a substrate of the enzyme, it can generally be made into an inhibitor of the target enzyme by modifying the region of the ligand that binds to the active site to prevent the ligand from being used as a substrate.

The above methods were utilized to evaluate a combinatorial library for PTP1B ligands and inhibitors. The library consisted of compound 3 (FIG. 1), wherein the linker components consisted of the 23 linkers 4-26 illustrated in FIG. 3, and the peripheral site-targeted components consisted of the 8 compounds A-H of FIG. 2. The library thus consisted of Compound 3 substituted with every combination of the 23 linkers and 8 peripheral site-targeted components (total number of library members=184).

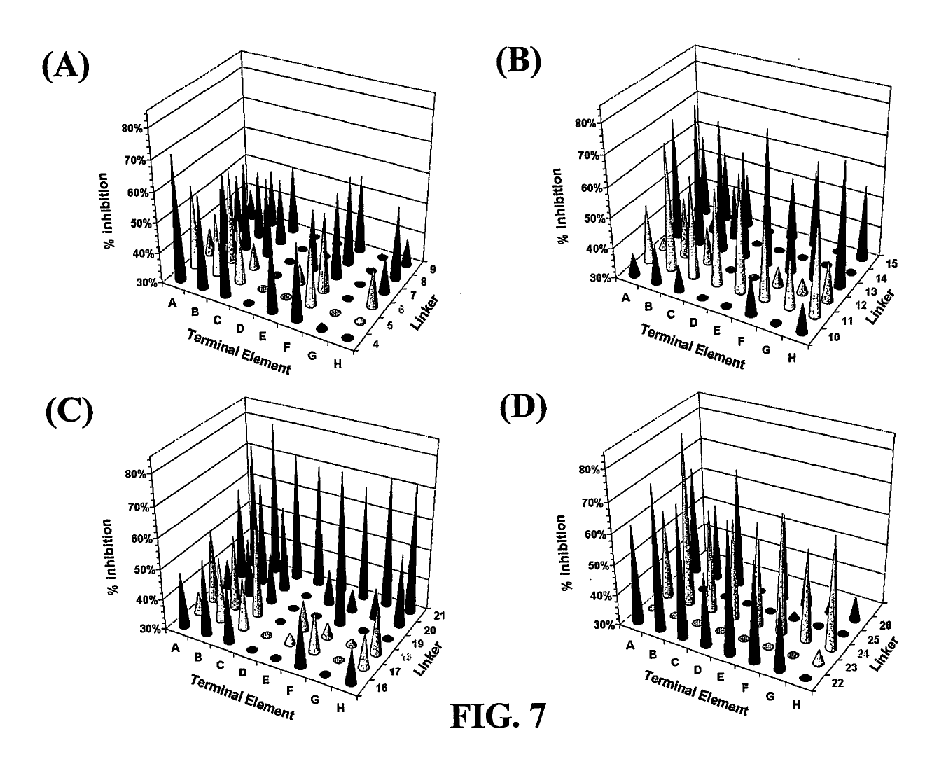

Each library member was tested for its ability to displace GST-PTP1B/C215S from bound DADEpYL. The results are provided in FIG. 7. The specific library components that were capable of inhibiting binding of GST-PTP1lB/C215S to DADEpYL by at least 30% (indicating ligand activity) were compound 3 consisting of the following linker components and peripheral site-targeted components: 4A, 4B, 4C, 4E, 4F, 5A, 5B, 5C, 5F, 6A, 6B, 6E, 6F, 6H, 7A, 7B, 7C, 7E, 7F, 7H, 8A, 8B, 8C, 8F, 8H, 9A, 9B, 9C, 9F, 9H, 10A, 10B, 10C, 10F, 10H, 11A, 11B, 11C, 11D, 11E, 11F, 11G, 11H, 12A, 12B, 12C, 12F, 12G, 12H, 13A, 13B, 13C, 13D, 13E, 13F, 13G, 13H, 14A, 14B, 14C, 15A, 15B, 15C, 15E, 15F, 15H, 16A, 16B, 16C, 16F, 16H, 17A, 17B, 17C, 17E, 17F, 17H, 18A, 18B, 18C, 18E, 18F, 18G, 18H, 19A, 19B, 19C, 19F, 20A, 20B, 20C, 20D, 10F, 20G, 20H, 21A, 21B, 21C, 21D, 21E, 21F, 21G, 21H, 22A, 22B, 22C, 22D, 22E, 22F, 22G, 23H, 24A, 24B, 24C, 24D, 24E, 24F, 24G, 24H, 25F, 26A, 26B, 26C, 26E, 26F, 26G, and 26H. Particularly effective were 21B and 24B; the most effective of the tested compounds was 21B. The skilled artisan would recognize from these results that some of the linker components and peripheral site-targeted components were more effective than other such components in forming a PTP1B ligand when present in compound 3. Specifically, linkers 11, 13, 21, 22 and 24 and peripheral site-targeted components A, B, C, F and H, particularly B, were the most effective components of compound 3 in forming a PTP1B ligand.

Based on the above information, the skilled artisan could identify, without undue experimentation, peripheral site-targeted components other than A-H that would likely be a component in a PTP1B ligand when combined with superior linkers 11, 13, 21, 22 and 24. In particular, such peripheral site-targeted components other than A-H that have an aromatic ring could be identified without undue experimentation. Also, the skilled artisan could identify, without undue experimentation, linker components other than 4-26 that would likely be a component in a PTP1B ligand when combined with peripheral site-targeted components A-H. Therefore, the PTP1B ligands envisioned as within the scope of the invention go beyond compound 3 with components 4-26 and A-H.

The present invention is thus also directed to a compound comprising an active site-targeted component, a linker component, and a peripheral site-targeted component, where the linker component is covalently bound to the active site-targeted component and the peripheral site-targeted component is covalently bound to the linker component, and wherein the active site-targeted component has the formula as in compound 3 of FIG. 1, and wherein the linker component and the peripheral site-targeted component are any organic molecule of less than 500 Dalton. Such compounds are useful, for example, in combinatorial libraries for discovering ligands of PTP1B. In preferred embodiments, the above compound comprises compound 3 of FIG. 1, where X and Y are independently any organic molecule of less than 500 Dalton. In other preferred embodiments, the linker component consists of carbon, oxygen, nitrogen and/or hydrogen and the peripheral site-targeted component has an aromatic ring and consists of carbon, oxygen, nitrogen, phosphorous, and/or hydrogen. Preferably, the compound is a ligand of PTPLB. In other preferred embodiments, the linker component is one of elements 4 through 26 of FIG. 3; more preferably elements 11, 13, 21, 22 or 24 of FIG. 3. Preferred peripheral site-targeted components are one of elements A through H of FIG. 2; more preferably elements A, B, C, F or H.

In related embodiments, the invention is directed to PTP1B ligands comprising the formula of compound 3 of FIG. 1. Preferably, the linker component and the peripheral site-targeted component are the following elements of FIGS. 3 and 2, respectively: 4A, 4B, 4C, 4E, 4F, 5A, 5B, 5C, 5F, 6A, 6B, 6E, 6F, 6H, 7A, 7B, 7C, 7E, 7F, 7H, 8A, 8B, 8C, 8F, 8H, 9A, 9B, 9C, 9F, 9H, 10A, 10B, 10C, 10F, 10H, 11A, 11B, 11C, 11D, 11E, 11F, 11G, 11H, 12A, 12B, 12C, 12F, 12G, 12H, 13A, 13B, 13C, 13D, 13E, 13F, 13G, 13H, 14A, 14B, 14C, 15A, 15B, 15C, 15E, 15F, 15H, 16A, 16B, 16C, 16F, 16H, 17A, 17B, 17C, 17E, 17F, 17H, 18A, 18B, 18C, 18E, 18F, 18G, 18H, 19A, 19B, 19C, 19F, 20A, 20B, 20C, 20E, 10F, 20G, 20H, 21A, 21B, 21C, 21D, 21E, 21F, 21G, 21H, 22A, 22B, 22C, 22D, 22E, 22F, 22G, 23H, 24A, 24B, 24C, 24D, 24E, 24F, 24G, 24H, 25F, 26A, 26B, 26C, 26E, 26F, 26G, and 26H. More preferably, the linker component is either element 21 or 24 of FIG. 3; most preferably element 21. The most preferred peripheral site-targeted component is element B of FIG. 2.

Any compounds comprising compound 3 that exhibits PTP1B ligand activity would be expected to be converted into a PTP1B inhibitor by substituting the phosphate group of the active site-targeted component with a diflorophosphonate group. It would also be expected that the PTP1B ligands with the highest affinity (as shown by the greatest ligand activity in the competitive assay previously described) would have the highest PTP1B inhibitory activity.

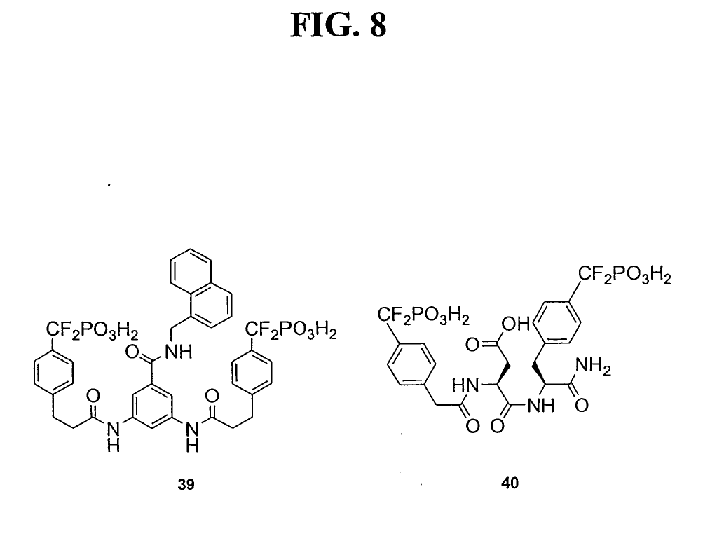

A particularly preferred inhibitor is compound 40 of FIG. 8, which is the most specific and the highest affinity inhibitor of PTP1B identified to date, having a Kᵢ value of about 2.4 nM (see Example 1).

Any of the above-described compounds, ligands or inhibitors can be made to have increased membrane permeability and superior ability to enter cells by further conjugating the compounds with any of a number of uncharged or positively charged moieties, for example a fatty acid moiety or a polyarginine moiety. See, e.g., Example 2. Thus, any of the above-described compounds, ligands or inhibitors, further comprising a fatty acid moiety or polyarginine moiety is envisioned as within the scope of the invention.

The fatty acid moiety is preferably at least 6 carbon atoms, more preferably at least 8, even more preferably at least 10, and most preferably 15 carbon atoms long. The polyarginine moiety preferably comprises at least 4 arginine, more preferably at least 6 arginines, and most preferably 8 arginines long.

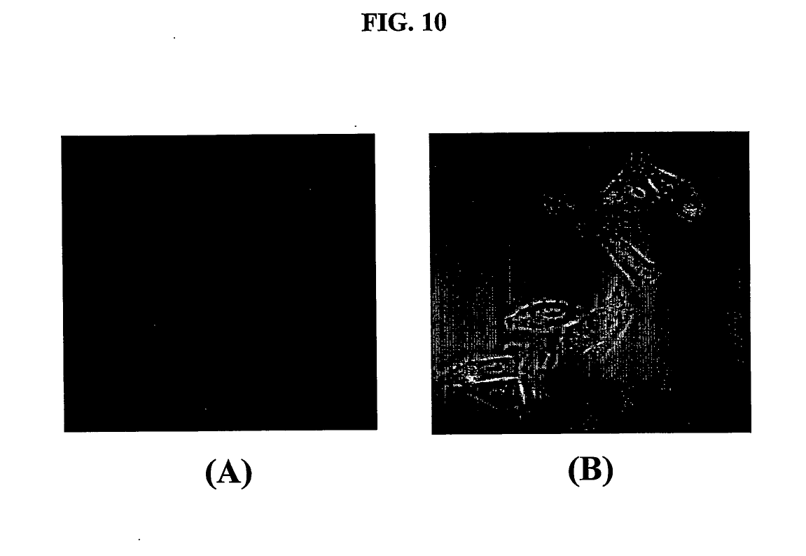

A detectable moiety can also usefully be conjugated to any of the above-described compounds, ligands or inhibitors to make the compound visable, e.g., in a micrograph of a cell treated with the compound (see FIG. 10) or in a cell fraction. Examples of such useful detectable moieties include a radioactive atom (e.g., ³²p, ¹⁴C, or ³H), a ligand or hapten that can be further detected with the corresponding binding partner or antibody (e.g., biotin, detectable with, e.g., radiolabeled avidin; digoxygenin, detectable with, e.g., peroxidase-labeled anti-digoxygenin antibody), or a fluorescent molecule, such as fluorescein or, more preferably, rhodamine.

Any of the identified PTP1B ligands, when converted into an inhibitor by substituting the phosphate group of the active site-targeted component with a diflorophosphonate group, would be expected to be useful in methods of preventing or treating obesity or Type II diabetes. The methods of preventing or treating obesity comprise administering any of the above-described inhibitors to a patient that is at risk for obesity or obese, respectively. The methods of preventing or treating Type II diabetes comprise administering any of the above-described inhibitors to a patient that is at risk for Type II diabetes, or has Type II diabetes, respectfully. Preferably, the inhibitor is in a pharmaceutically acceptable excipient. Such excipients are well known in the art and are generally chosen based on the route of administration that is desired. See below. In particularly preferred embodiments, the inhibitor is incorporated into liposomes, which enhance the ability of the inhibitor to pass through a cell membrane and into a cell, where it would be more likely to encounter PTP1B and provide a therapeutic benefit. In other preferred embodiments of these methods, the inhibitor further comprises a moiety facilitating entry into cells as previously discussed, for example a fatty acid moiety or a polyarginine moiety.

The route of administration and the dosage of the inhibitor to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Thus, depending on the condition, the inhibitor can be administered orally, parenterally, intranasally, vaginally, rectally, lingually, sublingually, bucally, intrabuccaly and transdermally to the patient.

Accordingly, inhibitor compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

Inhibitor compositions of the present invention can easily be administered parenterally such as for example, by intravenous, intramuscular, intrathecal or subcutaneous injection. Parenteral administration can be accomplished by incorporating the inhibitor compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the inhibitor in the glycerin, mixing the heated glycerin after which purified water may be added, and pouring the hot mixture into a suppository mold.

Transdermal administration includes percutaneous absorption of the inhibitor through the sidn. Transdermal formulations include patches (such as the well-known nicotine patch), ointments, creams, gels, salves and the like.

The present invention includes nasally administering to the mammal a therapeutically effective amount of the inhibitor. As used herein, nasally administering or nasal administration includes administering the inhibitor to the mucous membranes of the nasal passage or nasal cavity of the patient. As used herein, pharmaceutical compositions for nasal administration of a inhibitor include therapeutically effective amounts of the agonist prepared by well-known methods to be administered, for example, as a nasal spray, nasal drop, suspension, gel, ointment, cream or powder. Administration of the inhibitor may also take place using a nasal tampon or nasal sponge.

The present invention is also directed to methods of inhibiting the activity of a PTP1B, comprising contacting the PTP1B with any of the above-described PTP1B inhibitors. In preferred embodiments, the PTP1B inhibitor is compound 40 (FIG. 8) or an analog. In some embodiments of these methods, the PTP1B is in a living cell. In those embodiments, compounds 40A, 40B, or 40C are particularly preferred. Preferably, the cell is in a living vertebrate. In more preferred embodiments, the vertebrate is a mammal. In the most preferred embodiments, the vertebrate is a human.

Preferred embodiments of the invention are described in the following Examples. Other embodiments within the scope of the claims herein will be apparent to one skilled in the art from consideration of the specification or practice of the invention as disclosed herein. It is intended that the specification, together with the Example, be considered exemplary only, with the scope and spirit of the invention being indicated by the claims which follow the examples.

### EXAMPLE 1

### Acquisition of a Specific and Potent PTP1B Inhibitor from a Novel Combinatorial Library and Screening Procedure

Kinetic studies of PTPases with pTyr-containing peptides have previously showed that pTyr (e.g., the active site targeted component of compound 3 in FIG. 1) alone is not sufficient for high affinity binding and residues surrounding the pTyr contribute to efficient substrate recognition (12, 13). This suggests that there are sub-pockets bordering the active site that can be targeted to enhance inhibitor affinity and selectivity. Furthermore, the pTyr-binding site in PTPases is obviously smaller than the ATP site in protein kinases. Thus for PTPase inhibitor design, it is critical to consider adjacent peripheral sites in addition to the active site in order to gain potency and selectivity. This Example describes the construction of a novel combinatorial library designed to target both the active site and an adjacent peripheral site in PTP1B. Also described is the development of an ELISA-based affinity selection procedure that was used to screen for potent PTP1B ligands. A highly potent PTP1B inhibitor is identified (with a Kᵢ value of 2.4 nM) that exhibits several orders of magnitude selectivity in favor of PTP1B against a panel of PTPases. The following results demonstrate that it is feasible to achieve potency and selectivity for PTPase inhibition.

Materials and Methods

General Procedures.

All moisture-sensitive reactions were carried out in oven-dried glassware under a positive pressure of dry N₂ or Ar. DMA, DMF, DMSO, LHMDS, CH₂Cl₂, and THF for moisture-sensitive reactions were purchased from Aldrich in Sure/Seal™ bottles. All reactions were followed by TLC using E. Merck silica gel 60 F-254. Flash column chromatography was performed using J. T. Baker silica gel (230-400 mesh). BOP, DIC, HBTU, HOBt, piperidine, PyBOP, TFFH, and TSTU for peptide synthesis were purchased from Advanced ChemTech. The structures of new compounds were characterized by ¹H-NMR (300 MHz), ¹³C-NMR (75.5 MHz), ¹⁹F-NMR (282 MHz) and ³¹P-NMR (121 MHz) at 299 K unless otherwise indicated, and by ESI-MS analysis.

Peptide Synthesis.

Peptides (biotinyl-caproic acid-DADEpYL-amide and 7-hydroxycoumarin-caproic acid-DADEpYL-amide) were synthesized on Rink amide resin (Advanced ChemTech) using a standard protocol for HBTU/HOBt/NMM activation of Fmoc-protected amino acid derivatives (Advanced ChemTech or Novabiochem). 7-Hydroxycoumarin-4-acetic acid and biotin (Aldrich) were activated with 1.5 eq. TSTU and 4 eq. DIPEA in DMF. Side chains of Asp, and Glu were tert-butyl protected; the phosphate group of pTyr was mono-benzyl ester protected. The coupling reaction was performed in DMF for 1.5 h using a 3-fold excess of acid relative to resin-bound amine. Fmoc removal was performed with 20% piperidine in DMF. Final cleavage and side chain deprotection was achieved with 95% TFA and 2.5% TIS in water for 2 hr. The resin was removed by filtration, and the remaining solution concentrated. Dry diethyl ether was added and the precipitated peptides collected by centrifugation. The peptides were resuspended, washed twice with ether, dissolved in water, and purified by semi-preparative reverse phase HPLC. All peptides were obtained in high purity (>95%) as analyzed by MALDI-TOF MS and analytical HPLC.

Synthesis of PTP1B Ligand library.

The library was synthesized on a cystamine-modified Tentagel S NH₂ resin 1 using Fmoc chemistry (14) (FIG. 4). pTyr was attached to the amino terminus of the resin-linked cystamine (8 g). After Fmoc removal by two 5 min treatments with 30% piperidine in DMF, the resin was washed with DMF, CH₂Cl₂, isopropanol, and ether, and then the residual solvent removed in vacuo. The resin was distributed in 220 mg quantities into 20 mL polypropylene filtration tubes (Supelco) for coupling of the next component. The linking diversity elements 4-25 (FIG. 3) were incorporated (except for the absence of a diversity element 26) into the library in the Fmoc-protected form, which were either commercially available or prepared by treatment of commercially available amino acids with Fmoc-Osu in 1:1 THF/10% Na₂CO₃. Coupling was accomplished by one 2 hr and one 15 hr treatments with 6 eq. of the amino acid, 6 eq. of PyBOP, 6 eq. of HOBt, and 12 eq. of NMM in 4 mL DMF. The phosphate group of pTyr used in the library synthesis was mono-benzyl ester protected, and the acid side chains of Asp and Glu t-butyl ester protected. The N-terminal Fmoc group was deprotected by two 5 min treatments with 30% piperidine in DMF. The resin was then washed with DMF, CH₂Cl₂, isopropanol, and ether, and the residual solvent removed in vacuo. The coupling and deprotection steps were monitored by examination of free amine substitution level or Fmoc release during the course of the library synthesis until the coupling of the terminal diversity elements. The resin from each filtration tube was then distributed in 5.0 mg quantities into 8 wells in one line of the 96-well synthesis block. The terminal diversity elements A-H (FIG. 2) were incorporated into the library by one 2 hr and one 15 hr coupling using 6 eq. of the acid, 6 eq. of TFFH, and 12 eq. of DIPEA in 500 mL DMF. Those acids containing the phenyl phosphate group were prepared from the carboxyl methyl ester of the corresponding phenol via treatment with phosphoryl chloride in pyridine (15) followed by basic hydrolysis. 2,2′-bipyridine-4,4′-diacid was prepared from 4,4′-dimethyl-2,2′-bipyridine (GFS Chemicals) by treatment with KMnO₄ in 25% H₂SO₄ (16). Upon completion of the solid-phase assembly, side chain deprotection was accomplished by two 1 hr treatments with 90% TFA and 5% phenol in water. The resulting resin 3 (FIG. 1) was then washed extensively with CH₂Cl₂, DMF, MeOH, and H₂O before treatment with 10 mM DTT in 500 mL 50 mM Tris buffer (pH 8.0) for 3 hr. Finally the solution phase was filtered into the 96-well receiving plate to afford the spatially separated library members 3 at a concentration of 0.1 mM (assuming complete conversion for each member). Several library members were resynthesized on larger scale using the same procedure in high yield and purity (about 90%) as assessed by HPLC and MOLDI-TOP MS analysis. These library members include The structure 3 derived from subunits A and 17 (MOLDI-TOF MS calcd for [M] 653, found [M−H]⁻ 652.8) and structure 3 derived from subunits C and 6 (MOLDI-TOF MS calcd for [M] 633, found [M+H]⁺ 634.2).

Resynthesis of Selected High-Affinity PTP1B Ligands. Several high-affinity members of the library were selected based on the initial ELISA screening results, and their analogs without a thiol tail were synthesized on Rink resin according to the above peptide synthesis procedure. These compounds were again subjected to the ELISA evaluation and the highest-affinity compound having elements 21 and B was synthesized on large scale.

¹H-NMR (D₂O): d 7.4-7.2 (m, 8H), 4.76 (dd, J=6.0 Hz, 7.5 Hz, 1H), 4.68 (dd, J=5.7 Hz, 9.0 Hz, 1H), 3.66 (s, 2H), 3.27 (dd, J=5.7 Hz, 14 Hz, 1H), 3.04 (dd, J=9.0 Hz, 14 Hz, 1H), 2.9 (dd, J=6.0 Hz, 17 Hz, 1H), 2.7 (dd, J=7.5 Hz, 17 Hz, 1H); ¹³C-NMR (D₂O): d 175.8, 174.9, 174.3, 172, 151.2(d), 150.9(d), 132, 130.8, 130.74, 130.72, 121.06(d), 121.86(d), 54, 50, 41, 36, 35; ³¹P-NMR (D₂O): d −3.01, −3.03; MOLDI-TOF MS calcd for [M] 589, found [M+H]⁺ 590.

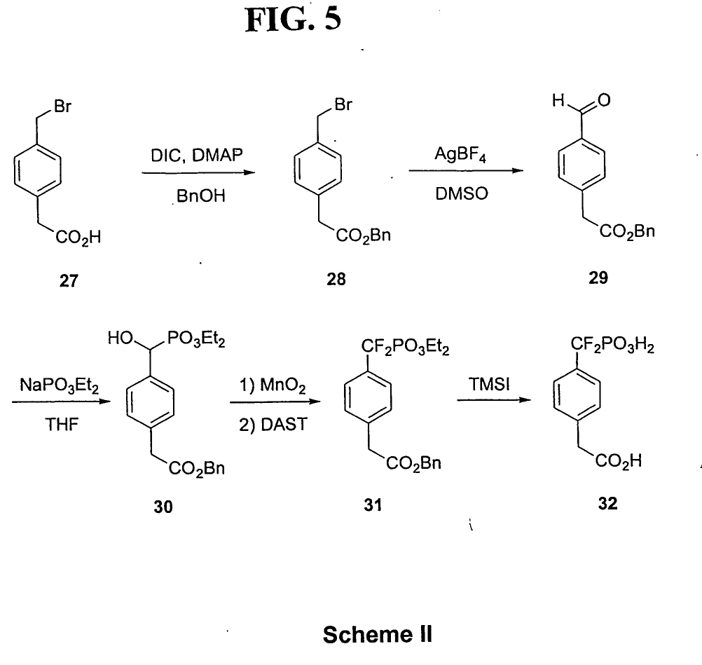

Synthesis of Benzyl 4-(Bromomethyl)phenylacetate (28). (FIG. 5) To a solution of 4-(bromomethyl)phenylacetic acid 27 (1.5 g, 6.55 mmol) in 30 mL CH₂Cl₂ was added benzyl alcohol (10 eq., 6.8 mL) and DMAP (0.05 eq., 40 mg). The solution was then chilled to 0° C. and DIC (520 mL, 0.5 eq.) was added in a dropwise fashion. The mixture was stirred at room temperature for 6 hr and then rotary evaporated to a reduced volume. Flash column chromatography yielded a white solid 28 (1.0 g, 96%). ¹H-NMR (CDCl₃): d 7.4-7.3 (m, 7H), 7.28 (d, J=7.9 Hz, 2H), 5.1 (s, 2H), 4.5 (s, 2H), 3.7 (s, 2H); ¹³C-NMR (CDCl₃): d 171, 137, 135, 134, 130, 129, 128.7, 128.5, 128.3, 67, 41, 33.

Synthesis of Benzyl 4-Formylphenylacetate (29). (FIG. 5) Silver tetrafluoroborate (17) (2.3 g, 11.8 mmol) was dissolved in dry DMSO (10 mL) and a solution of benzyl 4-(bromomethyl)phenylacetate 28 (3.0 g, 9.4 mmol) in dry DMSO (10 mL) was slowly added. The mixture was stirred at room temperature for 12 hr and then triethylamine (2 mL) was added. The mixture was kept for additional 15 min and then subjected to CH₂Cl₂/water extraction. The organic phase was concentrated via rotary evaporation and purified by flash column chromatography to afford a white solid 29 (1.96 g, 82%). ¹H-NMR (CDCl₃): d 10.0 (s, 1H), 7.8 (d, J=8.3 Hz, 2H), 7.5 (d, J=8.3 Hz, 2H), 7.3 (m, 5H), 5.1 (s, 2H), 3.8 (s, 2H); ¹³C-NMR (CDCl₃): d 192, 170, 140, 135.7, 135.6, 130.2, 130.1, 128.8, 128.6, 128.4, 67, 41.

Synthesis of Benzyl 4-[(Diethyphosphono)hydroxmethyl]phenylacetate (30) (FIG. 5). To sodium hydride (77 mg, 3.2 mmol) in 10 mL THF at −20° C. was added dropwise diethyl phosphite (430 mL, 3.3 mmol). The solution was stirred for 20 min before a solution of benzyl 4-formylphenylacetate 29 (750 mg, 3.0 mmol) in 8 mL THF was added. The solution was stirred for additional 30 min and the reaction quenched with 10 mL 5% NH₄Cl solution. The mixture was extracted by 3×15 mL ethyl acetate and the organic phase washed with,brine, dried over sodium sulfate, filtered, and concentrated by rotary evaporation. Subsequent flash column chromatography furnished a colorless oil 30 (820 mg, yield 71%).

¹H-NMR (CDCl₃): d 7.4 (dd, J=1.5 Hz, 7.9 Hz, 2H), 7.3-7.2 (m, 7H), 5.1 (s, 2H), 5.0 (d, J=11 Hz, 1H), 4.8 (s, broad, 1H), 4.0 (m, 4H), 3.6 (s, 2H), 1.23 (t, J=7.2 Hz, 3H), 1.19 (t, J=7.2 Hz, 3H); ¹³C-NMR (CDCl₃): d 171(d), 136.9 (d), 135.8, 133.6(d), 129(d), 128.6, 128.2, 128.1, 127(d), 70(d), 67, 63(m), 41, 16(d); ³¹P-NMR (CDCl₃): d 22.6.

Synthesis of Benzyl 4-[(Diethphosphono)difluoromethyl]phenylacetate (31) (FIG. 5). To a solution of benzyl 4-[(diethyphosphono)hydroxymethyl]phenylacetate 30 (100 mg, 0.26 mmol) in dry CH₂Cl₂ (5 mL), 260 mg activated MnO₂ (85%, 2.5 mmol) was added in one portion. The mixture was stirred for 24 hr and then filtered through acid-washed silica gel. The filtrate was rotary evaporated and dried in vacuo to afford the ketophosphonate intermediate as a colorless oil. Without further purification, the oil was chilled to 0° C. and 1 mL DAST (7.5 mmol) added dropwise. The solution was stirred at room temperature for 6 hr and then diluted by 10 mL CH₂Cl₂. The resulting solution was added slowly to 15 mL saturated Na₂CO₃ soluion at 0° C. The mixture was extracted by 3×10 mL CH₂Cl₂ and the combined organic layer washed by brine, dried over sodium sulfate, filtered, concentrated via rotary evaporation, and purified by flash column chromatography to afford 31 (45 mg, 43%). ¹H-NMR (CDCl₃): d 7.5 (d, J=7.9 Hz, 2H), 7.4-7.2 (m, 7H), 5.1 (s, 2H), 4.2 (m, 4H), 3.7 (s, 2H), 1.3 (t, J=7.2 Hz, 6H); ¹³C-NMR (CDCl₃): d 170, 137, 136, 132 (m), 129, 128.8, 128.7, 128.5, 128.4, 128.3, 128.2, 126(m), 115(m), 67, 65(d), 41, 16(d); ³¹P-NMR (CDCl₃): d 7.5 (t, J=116 Hz).

Synthesis of 4(Phosphonodifluoromethylphenylacetic Acid (32) (FIG. 5). Benzyl 4-[(diethyphosphono)difluoromethyl]phenylacetate 31 (123 mg, 0.3 mmol) was chilled to 0° C. by ice-water bath, and 1 mL of TMSI (7.0 mmol) was added to the reaction solution which was subsequently stirred at room temperature overnight. The solution was concentrated by rotary evaporation to an oily residue, dissolved in a mixed solution of 1 mL acetonitrile, 1 mL water and 0.5 mL TFA and stirred for 2 hr. The solution was then rotary evaporated to dryness, dissolved in water, and washed by ether. The aqueous solution was subjected to HPLC purification to afford the desired product 32 (28 mg, 35%). ¹H-NMR (D₂O): d 7.6 (d, J=7.9 Hz, 2H), 7.4 (d, J=7.9 Hz, 2H), 3.8 (s, 2H); ¹³C-NMR (D₂O): d 176, 136(d), 133(dt), 129, 126(dt), 120(dt), 40; ³¹P-NMR (D₂O): d 5.4 (t, J=105 Hz).

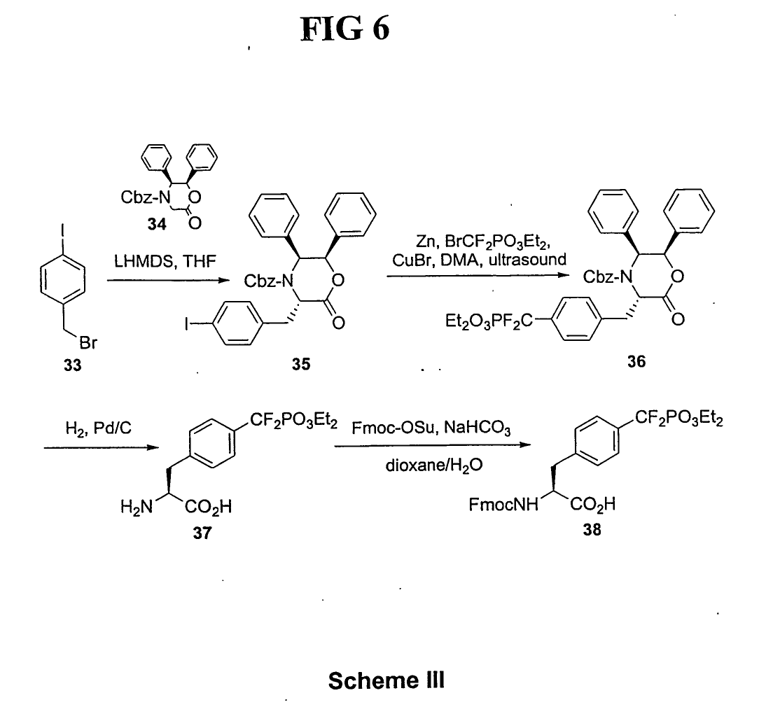

Synthesis of Benzyl (2R,3S)-6-Oxo-2,3-diphenyl-5-(4-iodobenzyl)-4-morpholinecarboxylate (34) (FIG. 6). 1 M LHMDS in THF (550 mL, 0.55 mmol) was added in a dropwise fashion to a solution of iodobenzyl bromide 33 (149 mg, 0.50 mol), the lactone 34 (213 mg, 0.55 mmol), and HMPA (1.5 mL) in THF (15 mL) at −78° C. (18). After string for 2 hr at −78° C., the mixture was diluted with EtOAc, washed with water and brine, dried over sodium sulfate, filtered, and the solvent removed via rotary evaporation. Flash column chromatography afforded the desired aryl iodide 33 (242 mg, 80%). Two conformers were observed in a ratio of 1:2 at 299 K; ¹H-NMR (CDCl₃): d major conformer 7.71 (d, J=7.9 Hz, 2H), 7.43-7.03 (m, 1 1H, overlapping), 6.83 (m, 2H, overlapping), 6.68-6.62 (m, 2H, overlapping), 6.53 (d, J=7.5 Hz, 2H), 5.35-5.25 (m, 2H, overlapping), 5.20-5.03 (m, 2H, overlapping), 4.91 (d, J=3.0 Hz, 1H), 4.51 (d, J=3.0 Hz, 1H), 3.63 (dd, J=6.8 Hz, 14 Hz, 1H), 3.47-3.31 (m, 1H, overlapping), minor conformer 7.63 (d, J=7.9 Hz, 2H), 7.43-7.03 (m, 11H, overlapping), 6.83 (m, 2H, overlapping), 6.72 (d, J=7.5 Hz, 2H), 6.68-6.62 (m, 2H, overlapping), 5.35-5.25 (m, 1H, overlapping), ), 5.23 (dd, J=3.4 Hz, 6.8 Hz, 1H), ), 5.20-5.03 (m, 3H, overlapping), 4.71 (d, J=3.0 Hz, 1H), 3.47-3.31 (m, 2H, overlapping); ESI-MS calcd for [M] 603, found [M+H]⁺ 604.

Synthesis of Benzyl (2R,3S)-6-Oxo-2,3-diphenyl-5-[(4-((diethylphosphono)difluoro-methyl)benzyl)]-4-morpholinecarboxylate (36) (FIG. 6). Zinc powder (520 mg, 8 mmol) in DMA (4 mL) was sonicated for 1 hr prior to treatment with a solution of diethyl bromodifluorophosphonate (1.42 mL, 8 mmol) in DMA (4 mL) (19). Sonication was continued for an additional 3 hr, and then cuprous bromide (1.15 g, 8 mmol) was added in one portion. After 30 min a DMA solution (4 mL) of the aryl iodide 35 (2.40 g, 4 mmol) was added dropwise, and the resulting mixture was stirred for 24 hr, diluted with EtOAc, washed with water and brine, dried over sodium sulfate, filtered, and the solvent removed via rotary evaporation. Flash column chromatography afforded the desired alkylated lactone 36 (1.38 g, 52%). Two conformers were observed in a ratio of 3:7 at 299 K; ¹H-NMR (CDCl₃, 299 K): d major conformer 7.82 (d, J=7.9 Hz, 2H), 7.61-7.23 (m, 11H, overlapping), 7.01 (d, J=7.9 Hz, 2H), 6.83 (d, J=7.9 Hz, 2H), 6.68 (d, J=7.9 Hz, 2H), 5.55-5.43 (m, 1H, overlapping), 5.33-5.17 (m, 2H, overlapping), 5.08 (d, J=3.0 Hz, 1H), 4.61 (d, J=3.0 Hz, 1H), 4.43-4.19 (m, 4H, overlapping), 3.93 (dd, J=6.8 Hz, 14 Hz, 1H), 3.75-3.55 (m, 1H, overlapping), 1.44 (m, 6H, overlapping), minor conformer 7.75 (d, J=7.9 Hz, 2H), 7.61-7.23 (m, 13H, overlapping), 6.88 (d, J=7.9 Hz, 2H), 6.78 (d, J=7.9 Hz, 2H), 5.55-5.43 (m, 1H, overlapping), 5.43 (dd, J=3.4 Hz, 6.8 Hz, 1H), 5.33-5.17 (m, 2H, overlapping), 4.85 (d, J=3.0 Hz, 1H), 4.43-4.19 (m, 4H, overlapping), 3.75-3.55 (m, 2H, overlapping), 1.44 (m, 6H, overlapping); 19F-NMR (CDCl₃, 299 K): d major conformer −108.58 (d, J=115 Hz), −108.78 (d, J=115 Hz), minor conformer −108.67 (d, J=115 Hz), −108.83 (d, J=115 Hz); ³¹P-NMR (CDCl₃, 299 K): d 7.2 (t, J=115 Hz); Conformers were not observed at 373 K; ¹H-NMR (DMSO, 373 K): d 7.5 (d, J=7.9 Hz, 2H), 7.4 (d, J=7.9 Hz, 2H), 7.3-7.0 (m, 11H), 6.9 (d, J=7.2 Hz, 2H), 6.6 (d, J=7.5 Hz, 2H), 5.8 (s, 1H), 5.2 (d, 3.0 Hz, 1H), 5.1 (dd, J=4.9 Hz, 8.3 Hz, 1H), 5.0 (s, 2H), 4.1 (m, 4H), 3.58 (dd, J=8.3 Hz, 14 Hz, 1H), 3.49 (dd, J=4.9 Hz, 14 Hz, 1H), 1.256 (t, J=7.2 Hz, 3H), 1.250 (t, J=7.2 Hz, 3H); ¹³C-NMR (DMSO, 373 K): d 167, 153, 138, 135.6, 135.4, 134, 129, 127.6, 127.5, 127.1, 126.9, 126.8, 125.8, 125.5(m), 115(m), 78, 67, 62(d), 60, 58, 15(d); 19F-NMR (DMSO, 373 K): d −105.9 (d, J=114 Hz), −106.2 (d, J=114 Hz); ³¹P-NMR (DMSO, 373 K): d 6.8 (t, J=114 Hz); ESI-MS calcd for [M] 663, found [M+H]⁺ 664.

Synthesis of 4-[Diethylphosphono]difluoromethyl]-L-phenylalanine (37) (FIG. 6). The alkylated lactone 36 (20) (478 mg, 0.72 mmol) in a small volume of MeOH was added to a suspension of 10% Pd/C (200 mg) in EtOH (4 mL) and THF (2 mL). The mixture was stirred for 24 hr under H₂ atmosphere and then filtered through Celite. The filtrate was rotary evaporated to dryness, triturated three times with ether and the residue then placed under vacuum to afford the desired amino acid 37 (252 mg, 100%). ¹H-NMR (CD₃OD): d 7.6 (d, J=7.9 Hz, 2H), 7.4 (d, J=7.9 Hz, 2H), 4.2 (m, 5H), 3.3 (dd, J=4.3 Hz, 14 Hz, 1H), 3.2 (dd, J=8.7 Hz, 14 Hz, 1H), 1.326 (t, J=7.2 Hz, 3H), 1.321 (t, J=7.2 Hz, 3H); ¹³C-NMR (CD₃OD): d 171, 139, 133(m), 131, 128, 117(m), 66(d), 55, 37, 16(d); ³¹P-NMR (CD₃OD): d 7.1 (t, J=118 Hz).

Synthesis of N-a-Fmoc-4-(Phosphonodifluoromethyl)-L-phenylalanine (38) (FIG. 6). A solution of the amino acid 37 (535 mg, 1.5 mmol) and NaHCO₃ (128 mg, 1.5 mmol) in water (5 mL) and dioxane (5 mL) was cooled in an ice bath and then treated with Fmoc-OSu (720 mg, 2.1 mmol) in a small amount of dioxane (20). After stirring for 3 hr at room temperature, the mixture was diluted with saturated NaHCO₃ (30 mL) and then washed with ether. The aqueous phase was acidified to pH 2 with 6 N HCl and extracted with EtOAc. The extracts were dried over sodium sulfate, filtered, and the solvents removed yielding the Fmoc amino acid 38 as a white solid (870 mg, 100%). The specific optical rotation [a]_{D}²⁴=44° (c=0.1 in chloroform) is consistent with previously reported values (20,21). ¹H-NMR (DMSO): d 7.9 (d, J=7.2 Hz, 2H), 7.7-7.3 (m, 10H), 4.2-4.0 (m, 8H), 3.1 (dd, J=4.5 Hz, 14 Hz, 1H), 2.9 (dd, J=11 Hz, 14 Hz, 1H), 1.18 (t, J=7.2 Hz, 3H), 1.17 (t, J=7.2 Hz, 3H); ¹³C-NMR (CDCl₃): d 173, 156, 144, 142, 139, 131(m), 130, 128, 127, 126, 125, 120, 115(m), 67, 65(d), 54, 47, 38, 16(d); ¹⁹F-NMR (CDCl₃): d −109 (d, J=118 Hz); ³¹P-NMR (CDCl₃): d 7.1 (t, J=118 Hz).

Synthesis of PTP1B Inhibitor Compound 40. (FIG. 8) Synthesis was performed on Rink amide resin using a standard protocol for HBTU/HOBt/NMM activation of acids. The coupling reaction was performed in DMF for 1.5 h using a 3-fold excess of acid relative to resin-bound amine. The fully protected Fmoc amino acid 38, the Fmoc protected Asp (with side chain tert-butyl protected), and the free acid 32 were sequentially coupled to the Rink amide resin. Fmoc removal after each coupling was effected with 20% piperidine in DMF. Final cleavage and side chain deprotection was achieved by treatment with 1 M TMSBr-thioanisole in TFA with 5% EDT and 1% m-cresol at 0° C. for 5 hr and then at room temperature for 16 hr. The resin was removed by filtration, and the remaining solution concentrated. The residue was triturated with ether, dissolved in water, and purified by semi-preparative reverse phase HPLC to afford the desired compound 40. ¹H-NMR (D₂O): d 7.6 (m, 4H), 7.4 (m, 4H), 4.7 (m, 2H), 3.7 (s, 2H), 3.3 (dd, J=5.7 Hz, 14 Hz, 1H), 3.1 (dd, J=9.4 Hz, 14 Hz, 1H), 2.9 (dd, J=6.0 Hz, 17 Hz, 1H), 2.7 (dd, J=7.9 Hz, 17 Hz, 1H); ¹³C-NMR (D₂O): d 175, 174.5, 174.4, 172, 139, 137, 133(m), 129.69, 129.63, 126(m), 115(m), 54, 50, 42, 37, 35; ¹⁹F-NMR (D₂O): d −108.58 (d, J=105 Hz), −108.66 (d, J=105 Hz); ³¹P-NMR (D₂O): d 5.36 (t, J=105 Hz), 5.35 (t, J=105 Hz); ESI-MS calcd for [M] 657, found [M−H]⁻ 656, [M+H]⁺ 658.

Subcloning of PTP1B/C215S to pGex-KG. The CDNA encoding the catalytic domain of human PTP1B (amino acid 1-321) was obtained using PCR from a human fetal brain cDNA library (Stratagene). The PCR primers used were 5′-AGCTGGATCCATATGGAGATGGAAAAGGAGTT (encoding both a BamHI and a NdeI site), and 3′-ACGCGAATTCTTAATTGTGTGGCTCCAGGATTCG (encoding a EcoRI site). The PCR product was digested with BamHI and EcoRI and subcolned into a pUC118 vector. The oligonucleotide primer used to convert Cys215 to Ser was 5′-TGGTGCACTCCAGTGCAGG-3′, where the underlined base indicates the base change from the naturally occurring nudeotide. The coding region for the PTP1B/C215S mutant was cut from pUC118-PTP1B/C215S with NdeI and EcoRI and ligated to the corresponding sites of plasmid pT7-7 (22). The coding region for PTP1B/C215S from pT7-7/PTP1B/C215S was cleaved with the restriction enzyme NdeI and sequentially treated with the Klenow fragment of DNA polymerase I to generate a blunt-ended molecule. The linearized DNA was digested again with restriction enzyme EcoRI. The vector pGEX-KG was cleaved with restriction enzymes SmaI (Blunt-ended) and EcoRI (cohesive-ended). The NdeI (blunt) to EcoRI DNA fragment of pT7-7/PTP1B/C215S containing PTP1B/C215S gene and the SmaI (blunt) to EcoRI fragment of pGex-KG encoding resistance to ampicillin were isolated and ligated together.

Protein Expression and Purification of GST-PTP1B and GST-PTP1B/C215S. pGex-KG/PTP1B (or PTP1B/C215S) was used to transform Escherichia coli BL21(DE3) by standard methods. Single colony was selected and grown in 10 mL of 2xYT medium containing 100 mg/mL ampicillin overnight with shaking at 37° C. A 10-mL overnight culture was transferred to 1 liter of 2xYT medium containing 100 mg/mL ampicillin and shaken at 37° C. until the absorbance at 600 nm was between 0.6-0.8. Following the addition of isopropyl-1-thio-b-D-galactopyranoside to a final concentration of 0.2 mM, the culture was incubated at 37° C. with shaking for an additional 4 hours. The cells were harvested by centrifugation at 5,000 rpm for 5 min, and the bacterial cell pellets were resuspended in 30 mL of PBS buffer (140 mM NaCl, 2.7 mM KCl, 10 mM Na₂HPO₄, 1.8 mM KH₂PO₄, pH 7.4) with 1 mM dithiothreitol, and 1% Triton X-100. The cells were lysed by passage through a French pressure cell press at 1200 p.s.i. twice. Cellular debris was removed by centrifugation at 15,000 rpm for 30 min, and the supernatant was decanted into a 50-mL conical tube, to which 2 mL of 50% slurry of glutathione-Sepharose 4B (Amersham Pharmacia Biotech) equilibrated with PBS buffer was added. After incubating with gentle agitation at 4° C. for 1 hr, the matrix was transferred to a column and washed by 10 bed volumes of PBS buffer with 1 mM dithiothreitol and 0.1% Triton X-100 and 5 bed volumes of 50 mM Tris, pH 7.5 and 1 mM dithiothreitol. After the column was left at room temerature for 10 miin, the fusion protein was eluted by addition of 1 bed volume of 10 mM reduced glutathione in 50 mM Tris, pH 8.0. The elution and collection steps were repeated five times. The eluents were pooled and concentrated with a Centriprep-30 filtration unit (Amicon) and changed to pH 7.0 buffer containing 50 mM 3′-3′-dimethylglutarate, 1 mM EDTA, 1 mM dithiothreitol, and I=0.15 M. The purified protein were made to 30% glycerol and stored at −20° C.

Other Recombinant PTPases. PTP1B (residues 1-321) (22), Yersinia PTPase (23), Stp1 (24), VHR (25), and MKP3 (26) were expressed in E. coli and purified as described previously. The coding sequence of the catalytic domain (amino acid residues 1-288) of the human T cell PTPase (TCPTP) was a generous gift from Dr. Harry Charbonneau and TCPTP was expressed and purified as described (27). Recombinant HePTP and the catalytic domains of SHP1 and SHP2 were expressed and purified as (His)₆-fusion proteins. The catalytic domains of PTPa, LAR and CD45 were expressed and purified as recombinant glutathione S-transferase (GST) fusion proteins (28). The intracellular fragment of PTPa, LAR and CD45 containing both of the PTPase domains was cleaved off the fusion protein as described using thrombin.

An ELISA-Based PTP1B Ligand Screening Procedure. To each well of a NeutrAvidin-coated 96-well microtiter plate was added 100 μL of 10 nM biotinyl-caproic acid-DADEpYL-amide in 50 mM 3,3-dimethyl glutarate, pH 7.0, I=0.15 M (DMG buffer). After incubation at 4° C. overnight, the plate was rinsed with the DMG buffer 3×(200 μL each). Each well was blocked with 100 μL of a solution containing 2% BSA and 0.2% Tween 20 in DMG buffer and shaken for 2 hours at room temperature. The wells were then rinsed with 4×200 μL of a solution containing 0.2% BSA, 0.1% Tween 20 in DMG, pH 7.0 buffer (BSA-T-DMG). In each well of a separate, uncoated 96-well plate, a 60 μL solution of the library component (500 nM in BSA-T-DMG) and a 60 μL solution of the GST-PTP1B/C215S fusion protein (0.4 nM in BSA-T-DMG) were mixed and incubated at room temperature for 1 hr. Then 100 μL of this mixture was added to each well of the blocked, biotinyl-caproic acid-DADEpYL-amide treated 96-well plate and the plate was shaken for 2 hr at room temperature. The wells were rinsed with 4×200 μL of a BSA-T-DMG. Polyclonal rabbit anti-GST antibody (100 μL, 100 ng/mL in BSA-T-DMG) was then added to each well and shaken for 1 hr at room temperature (or incubated overnight at 4° C.). The wells were washed with 4×200 μL of a BSA-T-DMG solution. To detect the amount of GST-PTP1B/C215S left in the well, horseradish peroxidase-conjugated mouse anti-rabbit antibody (100 μL, 200 ng/mL in BSA-T-DMG) was added to each well and shaken for 1 hr at room temperature. The wells were rinsed with 4×200 μL of a BSA-T-DMG and then 2×300 mL DMG buffer. 100 μL of peroxidase substrate (I-step Turbo TMB-ELISA, trimethylbenzidine) was added to each well and incubated for 5 to 30 min. To stop the peroxidase reaction, 100 μL of 1 M sulfuric acid solution was added to each well and the absorbance was measured at 450 nm with a SpectraMax 340 plate reader.

Determination of K_{d} Values. The coumarin-labeled pTyr-containing peptide 7-hydroxycoumarin-caproic acid-DADEpYL-amide is highly fluorescent and does not exhibit significant change in fluorescence upon PTP1B binding. Therefore, the K_{d} value for the binding of 7-hydroxycoumarin-caproic acid-DADEpYL-amide peptide to PTP1B/C215S was determined via equilibrium dialysis as previously described (14). All measurements were performed in 50 mM 3,3-dimethyl glutarate, pH 7.0, I=0.15 M buffer at 4° C. Briefly, Slide-A-Lyzer dialysis slide cassettes (Pierce, 10 kDa molecular weight cut-off, 0.1 to 0.5 mL capacity) were used which contained 100 nM GST-PTP1B/C215S and 100 nM 7-hydroxycoumarin-caproic acid-DADEpYL-amide. The cassettes (400 μl final volume) were placed in a beaker containing 100 mL of 100 nM 7-hydroxycoumarin-caproic acid-DADEpYL-amide in the same buffer. As a consequence, the concentration of non-PTP1B-bound peptide was held constant in the dialysis slide cassette over the course of the dialysis experiment (16 hrs). Differences in fluorescence between the solution in the slide cassette and that in the beaker were determined. The excitation wavelength for the coumarin peptide was 325 nm and the emission was monitored at 460 nm. The K_{d} value was calculated from equation 1:

$${K}_{p} = \frac{( [ E ] - [ E \cdot P ] ) [ P ]}{[ E \cdot P ]} \qquad ( Eq . 1 )$$

where Kₚ=K_{d} of 7-hydroxycoumarin-caproic add-DADEpYL-amide for PTP1B/C215S, [E]=total PTP1B/C215S concentration, [P]=total 7-hydroxycoumarin-caproic acid-DADEpYL-amide concentration, and [E·P]=concentration of 7-hydroxycoumarin-caproic acid-DADEpYL-amide bound to PTP1B/C215S.

A competition-based assay was used to determine the K_{d} value for the binding of the non-fluorescent compound 21B to PTP1B/C215S. The cassettes (400 μl final volume) contained 390 nM GST-PTP1B/C215S, 248 nM non-fluorescent high-affinity PTP1B ligand 21B, and 3.97 μM 7-hydroxycoumarin-caproic acid-DADEpYL-amide. The cassettes were placed in a beaker containing 100 mL of 248 nM non-fluorescent high affinity PTP1B ligand 21B and 3.97 μM 7-hydroxycoumarin-caproic acid-DADEpYL-amide. The K_{d} for compound 21B was obtained via competitive displacement of the coumarin derivative using equation 2 (14):

$${K}_{L} = \frac{{K}_{p} \frac{[ L ] [ E \cdot P ]}{[ P ]}}{[ E ] - {K}_{p} \frac{[ E \cdot P ]}{[ P ]} - [ E \cdot P ]} \qquad ( Eq . 2 )$$

where K_{L}=K_{d} of 21B for PTP1B/C215S, Kₚ=K_{d} of 7-hydroxycoumarin-caproic acid-DADEpYL-amide for PTP1B/C215S, [E]=total PTP1B/C215S concentration, [P]=total 7-hydroxycoumarin-caproic acid-DADEpYL-amide concentration, [L]=total 21B concentration, and [EP]=concentration of 7-hydroxycoumarin-caproic acid-DADEpYL-amide bound to PTP1B/C215S.

Determination of Inhibition Constants (Kᵢ) and IC₅₀ Values. The PTPase activity was assayed using p-nitrophenyl phosphate (pNPP) as a substrate at 25° C. in 50 mM 3,3-dimethylglutarate buffer, pH 7.0, containing 1 mM EDTA with an ionic strength of 0.15 M adjusted by addition of NaCl. The reaction was initiated by the addition of the enzyme to a reaction mixture (0.2 mL) containing various concentration of pNPP and quenched after 2-3 min by addition of 0.05 mL of 5 N NaOH. The range of substrate concentration used was 0.2-5 Kₘ. The nonenzymatic hydrolysis of the substrate was corrected by measuring the control without addition of enzyme. After quenching, the amount of product p-nitrophenol was determined from the absorbance at 405 nm detected by a Spectra MAX340 microplate spectrophotometer (Molecular Devices) using a molar extinction coefficient of 18,000 M⁻¹cm⁻¹. The Michaelis-Menten kinetic parameters were determined from a direct fit of the velocity versus substrate concentration data to Michaelis-Menten equation using the nonlinear regression program KinetAsyst (IntelliKinetics, State College, Pa.). Inhibition constants for the PTPase inhibitors were determined for PTP1B and TCPTP in the following manner. The initial rate at eight different substrate concentration concentrations (0.2 Kₘ to 5 Kₘ) was measured at three different fixed inhibitor concentrations (15). The inhibition constant was obtained and the inhibition pattern was evaluated using a direct curve-fitting program KINETASYST (IntelliKinetics, State College, Pa.). IC₅₀ values for various phosphatases were determined at 2 mM pNPP concentration.

Results and Discussion

As noted in the Introduction, biochemical and genetic studies suggest that PTP1B is a major modulator of insulin sensitivity and fuel metabolism. Thus PTP1B represents a potential therapeutic target for the treatment of Type II diabetes and obesity. Consequently, small molecules designed to inhibit PTP1B not only hold promise as pharmaceutical agents but also could function as probes for elucidating the roles of PTP1B in specific intracellular pathways involved in normal cellular processes. However, one major concern is that since the active site (i.e., pTyr binding site) is highly conserved among the large number of PTPases, the probability of obtaining inhibitors that selectively target one PTPase seems quite low. Nevertheless, the most effective approach for PTPase inhibitor design targets the active site.

Kinetic studies with pTyr-containing peptides showed that pTyr alone is not sufficient for high affinity binding by PTPases and residues surrounding the pTyr contribute to efficient substrate recognition (13, 29, 30). This suggests that there are sub-pockets bordering the active site that can be targeted to enhance inhibitor affinity and selectivity. Furthermore, active site specificity studies indicate that PTPase active sites possess significant plasticity such that a range of aryl phosphates with distinct functionalities can be accommodated within the catalytic/pTyr binding pocket (15, 31). We have found that, although nonpeptidic aryl phosphates are generally much poorer substrates than the pTyr-containing peptides, appropriately functionalized aromatic phosphates can exhibit Km values in the low μM range and are hydrolyzed by PTP1B as efficiently as the best peptide substrates reported for this enzyme (31). For example, bis-(para-phosphophenyl) methane (BPPM) is one of the best low-molecular weight nonpeptidic substrates identified for PTP1B (k_{cat}=6.9 s⁻¹, Kₘ=16 μM).

The crystal structure of PTP1B/C215S complexed with BPPM showed that BPPM binds, as expected, at the active site, and provided structural explanations for the higher affinity of BPPM relative to pTyr (22). Quite unexpectedly, the crystal structure revealed the presence of a second aryl phosphate-binding site positioned adjacent to the active site. This second site lies within a region that is not conserved among PTPases. As a consequence, this unanticipated observation suggested an alternative paradigm for the design of potent and specific PTP1B inhibitors; namely bidentate ligands that bind to both the active site and a unique adjacent peripheral site. In addition to the second aryl phosphate binding pocket, other sub-sites, positioned within the local vicinity of the active site, may also be conscripted for inhibitor design. For example, structures of PTPase in complex with pTyr-containing peptides and PTPase sequence alignments have suggested that the a1-b1 loop, the b5-b6 loop, the a5-a6 loop, and the WPD loop contain variable residues that may contribute to substrate specificity. Thus, our strategy to develop potent and PTPase-selective inhibitors for individual members of the PTPase family is to tether together two small ligands that are individually targeted to the active site and a unique proximal noncatalytic site. The rationale for the enhanced affinity of bidentate inhibitors is based on the principle of additivity of free energy of binding. The interaction of an inhibitor with two independent sites (e.g., pTyr site and a unique peripheral site) on one PTPase would be expected to confer exquisite specificity, since other PTPases may not possess an identical second site interaction. In the following, we describe a combinatorial approach for the identification of a highly potent and selective PTP1B inhibitor that is able to simultaneously occupy both the active site and a unique second site on PTP1B.

Library Design and Construction. Our first-generation library was designed to contain two linked motifs, one targeted to the pTyr-binding catalytic site, and the other targeted to a unique adjacent noncatalytic site in PTPLB. Due to the demanding synthetic requirements associated with the preparation of nonhydrolyzable phosphonate analogs (vide infra), we felt it prudent to prepare a library of synthetically accessible phosphate-based derivatives. Once a high affinity lead from the latter is identified, it can then be converted into an inhibitor by replacing the phosphate moiety with a difluorophosphonate group. Since pTyr is the canonical ligand for PTPase active site, we decided to structurally bias the library with pTyr in order to direct library members to the active site. A small array of structurally disparate aryl acids (A-H) (FIG. 2) were chosen and linked to pTyr in order to access binding interactions removed from the active site. These aryl acids include three phenylphosphate-containing species (A-C), three phenol-containing species (D-F), and two additional aromatic species (G-H). Members of the aryl acid array were separately linked to pTyr either directly (26) or via twenty-two different amino acids (4-25) (FIG. 3), which include nine linear aliphatic species (4-10, 15, 23), eleven ring-containing species (11-14, 16-20, 24-25), and two natural acidic amino acids (21-22). Inclusion of hydrophobic and charged amino acids as linkers could potentially provide additional interactions to enhance PTP1B binding. With these substructures, we constructed a synthetic library of 184 members [(pTyr) 1×(linkers) 23×(diversity elements) 8] by solid phase parallel synthesis (Scheme I, FIG. 4) using established approaches (see Materials and Methods and the references therein).

The library was synthesized on a disulfide-modified Tentagel S NH₂ resin 1 using Fmoc chemistry (14). The disulfide linkage between the peptide and the TentaGel resin is stable to the conditions of Fmoc-based solid phase peptide synthesis. Furthermore, the disulfide moiety is cleaved in essentially quantitative yield by conditions (i.e. DTT in buffer) that are compatible with standard enzyme assays, including the ELISA-based screen for PTP1B (vide infra). The pTyr was attached to the amine termini of cystamine as the starting building block. The resin was then split into equal portions for the separate coupling of the linkers 4-26. The resin from each linker-based reaction was subsequently distributed in 5.0 mg quantities into 8 wells of a single row of 96-well microplates. The terminal diversity elements A-H were then incorporated into the library. The resulting resin-linked library members 2 were extensively washed and then subsequently cleaved with 10 mM DTT in 500 mL 50 mM Tris buffer (pH 8.0) for 3 hr. The solution phase was vacuum filtered into a 96-well receiving plate to afford the spatially discrete library of 3 at a concentration of 0.1 mM (assuming complete conversion for each member). Several library members were resynthesized on a larger scale using the same procedure in high yield and purity (about 90%) as assessed by HPLC and MOLDI-TOF MS analysis.

Assay Development. The members of the synthetic library are aryl phosphates and therefore can potentially serve as PTPase substrates. One can identify efficient PTPase substrates by phosphatase activity-based assay (13, 31). However, the most efficient substrate, characterized by the highest k_{cat}/_{Km} value, does not necessarily possess the highest affinity for the enzyme. Our goal was to identify high-affinity PTP1B-binding ligands that can be subsequently converted into nonhydrolyzable analogs as PTP1B inhibitors. Thus we required an affinity-based assay that could easily be adopted for high-throughtput screening of a moderate size library of compounds. To this end, we developed an enzyme-linked immunosorbant assay (ELISA) to screen for high-affinity PTP1B substrates that avoids phosphate hydrolysis of library members by PTP1B. This assay requires the use of a catalytically deficient mutant PTP1B that retains the wild type binding affinity. We have previously shown that the active site Cys to Ser PTPase mutant has no measurable phosphatase activity (32) and that the PTP1B/C215S mutant exhibits similar affinity for substrates as the wild-type enzyme (33). We have also shown that the hexameric pTyr-containing peptide DADEpYL-amide is a high affinity PTP1B substrate (30, 33). We prepared the biotinyl-caproic acid-DADEpYL-NH₂ peptide and found that it displayed kinetic parameters similar to those reported for the DADEpYL-NH₂ peptide with the wild-type PTP1B (data not shown). Thus, in this assay the binding affinity of the library members was assessed by their ability to compete with the biotinylated phosphopeptide for binding to PTP1B/C215S.

In the ELISA-based assay (for details, see Materials and Methods), NeutrAvidin (or. streptavidin)-coated 96-well microtiter plates were first treated with 10 nM biotinyl-caproic acid-DADEpYL-NH₂ peptide. The plates were then blocked with a solution containing 2% BSA and 0.2% Tween 20 and rinsed with a buffer solution. Subsequently, members of the synthetic library (250 nM), individually incubated with GST-PTP1B/C215S (0.2 nM) were introduced into each well of the biotinyl-caproic acid-DADEpYL-NH₂ peptide treated plates. After extensive washing steps, the amount of GST-PTP1B/C215S bound to the biotinylated peptide was detected by primary polyclonal rabbit anti-GST antibody and secondary horseradish peroxidase-conjugated mouse anti-rabbit IgG antibody.

There are several key points to be noted concerning the ELISA-based assay. First, since the reference ligand (biotinylated DADEpYL-NH₂) is known to bind to the PTP1B/C215S active site (7), compounds that displace the reference ligand from PTP1B/C215S most likely bind to the active site as well. Second, since the catalytically inactive PTP1B/C215S binds ligands with equal potency as the wild-type enzyme, this assay furnishes a true assessment of the PTP1B binding ability of the library members. Third, it is known that the invariant active site Cys residue is essential for PTPase catalytic activity (8). Consequently, PTPases are prone to inactivation by oxidizing reagents and alkylating compounds. This has presented a serious problem for the PTPase activity-based inhibitor screening projects in which hits are identified based on the ability of the compounds to reduce the PTPase activity. The substitution of the active site Cys by a Ser (e.g., PTP1B/C215S) renders the mutant PTPase less sensitive to oxidation and alkylation and thus will likely eliminate “false” positives due to interactions with the active site Cys that destroy the phosphatase activity. Finally, since the assay is ELISA-based, it can be easily implemented for high-throughput PTPase inhibitor discovery.

Identification of High-Affinity PTP1B Substrates. The ELISA-based screening protocol employed library members fixed at a 250 nM concentration and was performed in duplicate. This affinity-based screen allowed us to identify several lead compounds that effectively displace GST-PTP1B/C215S from the biotinylated DADEpYL-NH₂, peptide. Several key points are clear from the results graphically depicted in FIG. 7. First, the naturally occurring amino acids 11, 13, 21, 22, and 24 serve as the most effective linkers. For example, all the Asp-containing library members (21A-21H in FIG. 7C) display significant inhibitory potency. Interestingly, these lead linkers are a mix of hydrophobic (11, 13, 24) and negatively charged (13, 21, 22) residues. The linker position is equivalent to the P-1 position (i.e. on the amino side of pTyr) in active site-directed PTPase peptide/protein substrates. We have previously shown that PTP1B undergoes distinct conformational changes that allow it to accommodate either hydrophobic or negatively charged residues at the P-1 site (9). Second, two of the most effective PTP1B ligands (21B and 24B) contain the same N-terminal element, the phosphorylated phenylacetic acid moiety B. Finally, PTP1B is dearly quite sensitive to the structural nature of the N-terminal element given the fact that closely related elements (A and C) which differ by a single methylene group are less effective than the lead B.

In order to obtain a more accurate assessment of the affinity of these compounds for PTP1B/C215S, we measured the IC₅₀ values (compound concentrations that block 50% of the ELISA readout at 450 nm) of the lead compounds (21B and 24B) using 39 as a reference (FIG. 8). For comparison, we also measured the IC₅₀ values of compounds 4A and 4B, which were less effective than 21B and 24B in displacing biotinylated DADEpYL-NH₂ from PTP1B/C215S (FIG. 7). To avoid potential problems associated with the possible oxidation of the thiol tail in the library compounds, we resynthesized compounds 4A, 21B, and 24B without the thiol tail. Table 1 lists the ratio of the IC₅₀ values of the test compounds relative to that of the reference compound 39. Since 39 is an established competitive inhibitor for PTP1B with a Kᵢ value of 1 mM (28), this IC₅₀ ratio should reflect the true affinity of the test compounds for PTP1B (in units of mM). As can be seen from Table 1, the presence of the thiol tail in the compounds does not affect the affinity of these compounds for PTP1B/C215S. It can be concluded that compounds 21B and 24B display binding affinities significantly higher than that of 39. In addition, compounds 21B and 24B also exhibit higher affinity for PTP1B than that of 4A and 4B, consistent with the ELISA results obtained at a single compound concentration (250 nM) (FIG. 7). Finally, although PTP1B can accommodate both Tyr (24) and Asp (21) at the P-1 position (9, 13), it appears that in the context of the terminal element B, the linker Asp (21) is slightly favored over Tyr (24).

TABLE 1
Relative Binding Affinity of Lead Compounds Determined by
the ELISA Assay

| Compound | IC₅₀(test)/IC₅₀(reference) |
|---|---|
| 39 | 1.0 |
| 4B | 0.70 |
| 4A (thiol tail eliminated) | 0.79 |
| 4A | 0.47 |
| 24B (thiol tail eliminated) | 0.050 |
| 24B | 0.043 |
| 21B (thiol tail eliminated) | 0.025 |
| 21B | 0.035 |

Determination of K_{d} Values. The intrinsic fluorescence associated with the N terminus appended coumarin moiety in the 7-hydroxycoumarin-caproic acid-DADEpYL-NH₂ peptide was not significantly altered in the presence of GST-PTP1B/C215S. This property enabled us to determine the dissociation constant for the coumarin derivative via equilibrium dialysis using Slide-A-Lyzer cassettes (see Materials and Methods). The K_{d} value for the binding of 7-hydroxycoumarin-caproic acid-DADEpYL-NH₂ to PTP1B/C215S is 420±20 nM at pH 7.0 and 4° C. This is similar to the K_{d} value for the binding of Ac-DADEpYL-NH₂ to PTP1B/C215S determined by isothermal titration calorimetry (800±100 nM) at pH 7.0 and 25° C. Using the same procedure, the K_{d} value for the lead compound 21B can be determined from its ability to displace the 7-hydroxycoumarin-caproic acid-DADEpYL-NH₂ peptide in the dialysis experiment. The K_{d} value for compound 21B furnished by equilibrium dialysis is 32±5 nM, which is in agreement with the affinity determined by the ELISA assay (Table 1, ˜30 nM).

Acquisition of a Nonhydrolyzable Derivative of 21B. Compound 40. As described above, we have identified the compound having elements 21B as the most potent PTP1B-binding ligand from a 184 member spatially discrete library. We next evaluated whether a nonhydrolyzable analog of 21B can serve as a potent and selective PTP1B inhibitor. Burke and his colleagues have shown that the aryl phosphate group in PTPase substrates can be replaced with a hydrolytically resistant difluorophosphonate moiety to produce effective PTPase inhibitors (34, 35). For example, when phosphonodifluoromethyl phenylalanine (F₂Pmp) replaces the pTyr in the hexapeptide DADEpYL-NH₂, the Kᵢ for the resulting peptide bearing F₂Pmp (200 nM for PTP1B) is over 1000 times more potent than the same peptide containing phosphonomethyl phenylalanine (Pmp) (33, 34, 36). This has been attributed to a direct interaction between the fluorine atoms and PTP1B active site residues (36). Thus we decided to replace the ester oxygens in 21B with the difluoromethylene group.

The corresponding nonhydrolyzable analog (40, FIG. 8) of the high affinity phosphomonoester (21B) was prepared via solid phase synthesis using the difluorophosphonate-containing derivatives 32 and 38. The hydrolytically resistant difluorophosphonate analog (32) of B was prepared from 4-(bromomethyl)phenylacetic acid as outlined in scheme II (FIG. 5) (28). The unnatural amino acid 38 was synthesized as illustrated in scheme III (FIG. 6). The diphenyloxyazinone intermediate 36 has been previously prepared in 5 steps from commercially available α-bromo-p-toluic acid acid in an overall 28% yield (20). We developed a somewhat more efficient synthesis (2 steps, 42% yield), utilizing the CuBr-mediated coupling of (diethoxyphosphinyl)difluoromethyl]zinc bromide (19) with the aryl iodide 35. The latter was obtained via the diastereoselective alkylation of the enolate of 34 with the commercially available iodobenzylbromide 33. The NMR of 36 (T=100° C.) revealed only a single diasatereomer, consistent with the high ee's previously reported for this method (18). Compound 36 was then converted to the desired Fmoc-protected amino acid 38 via hydrogenolysis and subsequent Fmoc protection (20). The standard rotation of 38 ([a]_{D}=44°; c=0.1 in CHCl₃) corresponds closely to previously reported values for this compound, confirming that the alkylation of 34 proceeded with high stereoselectivity. Compound 40 was subsequently assembled via the sequential addition of 38, Fmoc-Asp(O-tBu), and 32 to the Rink amide resin under standard solid phase Fmoc conditions.

Compound 40 Is the Most Potent and Specific PTP1B Inhibitor Identified to Date. The effect of the hydrolytically resistant compound 40 on the PTP1B-catalyzed pNPP hydrolysis reaction was examined at 25° C. in a pH 7.0, 50 mM 3,3-dimethylglutarate buffer, containing 1 mM EDTA and an ionic strength of 0.15 M (for details see Materials and Methods). Compound 40 inhibits the PTP1B reaction reversibly and the mode of inhibition is competitive with respect to the substrate (data not shown). The Kᵢ value for the inhibition of PTP1B by 40 is 2.4±0.2 nM. PTP1B inhibitors with Kᵢ or IC₅₀ values in the low nM range have been previously reported (37, 38). However, those measurements were conducted at low, and therefore nonphysiological, ionic strengths. Due to the electrostatic nature of the interactions between the inhibitors and PTP1B active site, it is possible that measurements at low ionic strength may overestimate the binding affinity of these compounds. In support of this, we note that the Kᵢ and K_{d} values of the hexapeptide DADE(F₂Pmp)L-NH₂ for PTP1B measured under our physiologically relevant conditions (ionic strength of 0.15 M) by enzyme inhibition and isothermal titration calorimetry, are 250 and 240 nM, respectively (33). By contrast, the Kᵢ value for the same peptide obtained under previously reported low ionic strength conditions (pH 7.3 in 50 mM Hepes, 5 mM DTT and 10 mg/mL BSA buffer) is 26 nM (37). In addition, the IC₅₀ value of the same peptide under similar low ionic strength conditions (pH 6.3, in 50 mM Bis-Tris, 2 mM EDTA, and 5 mM DTT buffer) is 30 nM (38). Since the ionic strength in both cases is much lower than 0.15 M it is understandable why a discrepancy exists in the reported PTP1B affinities of the hexapeptide. To further demonstrate the importance of salt concentration on the apparent binding affinity, we also measured the Kᵢ value of compound 40 under identical low salt conditions used by other groups. We found that the Kᵢ of 40 for PTP1B is 0.14±0.01 nM when measured at pH 7.3 in 50 mM Hepes, 5 mM DTT and 10 mg/mL BSA buffer. Similarly, the Kᵢ of 40 for PTP1B is 0.63±0.09 nM when measured at pH 6.3 in 50 mM Bis-Tris, 2 mM EDTA, and 5 mM DTT buffer. These results highlight the importance of controlling assay ionic strength to ensure meaningful comparison of inhibitory properties of PTPase ligands. Collectively, our results indicate that compound 40 is the most potent PTP1B inhibitor identified to date.

To determine if compound 40 is specific for PT?1B, the inhibitory activity of 40 toward a panel of protein phosphatases was evaluated. These included the nonreceptor-like, cytosolic PTPases: the Yersinia PTPase. TCPTP, HePTP, SHP1 and SHP2, the receptor-like PTPases: LAR, PTPa, and CD45, the dual specificity phosphatases: VHR, MKP3, and Cdc25A, the low molecular weight phosphatase Stp1, and the Ser/Thr protein phosphatase PP2C. Although a number of potent PTP1B inhibitors have been reported (28, 37-41), achieving selectivity, particularly between PTP1B and TCPTP, has been a considerable challenge. As shown in Table 2, compound 40 is highly selective for PTP1B, exhibiting a greater than three orders of magnitude preference for PTP1B versus nearly all phosphatases examined. More importantly, compound 40 also displays >10-fold selectivity in favor of PTP1B over TCPTP, which is the closest structural homologue of PTP1B (the catalytic domain of PTP1B [residues 1-279] is 69% identical and 85% homologous to that of TCPTP). The high selectivity that is observed for compound 40 without any further optimization is quite impressive, considering the general lack of selectivity that has been observed for inhibitors of the PTPase family members. These results demonstrate that it is possible to achieve both potency and selectivity in PTPase inhibitor development.

TABLE 2
Selectivity of Compound 40 against a Panel of Protein Phosphatases

| Phosphatase | Inhibition Potency |
|---|---|
| PTP1B | Kᵢ = 2.4 nM |
| TCPTP | Kᵢ = 26 nM |
| Yersinia PTP | IC₅₀ = 1.6 μM |
| SHP2 | IC₅₀ = 10 μM |
| SHP1 | IC₅₀ = 11 μM |
| LAR | IC₅₀ = 72 μM |
| HePTP | No inhibition at 10 μM |
| PTPα | No inhibition at 10 μM |
| CD45 | No inhibition at 10 μM |
| VHR | No inhibition at 10 μM |
| MKP3 | No inhibition at 10 μM |
| Cdc25A | No inhibition at 10 μM |
| Stp1 | No inhibition at 10 μM |
| PP2Cα | No inhibition at 10 μM |

Conclusions. In summary, we have described the parallel synthesis of a library of aryl phosphates designed to simultaneously occupy both the PTPase active site and an adjacent non-conserved peripheral site. An affinity-based ELISA screening procedure using the catalytically inactive PTP1B/C215S mutant led to the identification of a potent PTP1B-binding ligand, compound 21B. Conversion of 21B into its nonhydrolyzable difluorophosphonate analog 40 produced the most potent and selective PTP1B inhibitor reported to date. This result serves as a proof-of-concept in PTPase inhibitor development, as it demonstrates the feasibility of acquiring potent, yet highly selective, PTPase inhibitory agents. PTPase inhibitors, such as 40, should not only prove useful in dissecting the precise roles played by specific PTPases in signal transduction pathways, but should furnish a molecular foundation upon which therapeutically useful agents will be based.

### EXAMPLE 2

### Biological Effects of PTP1B Inhibitors

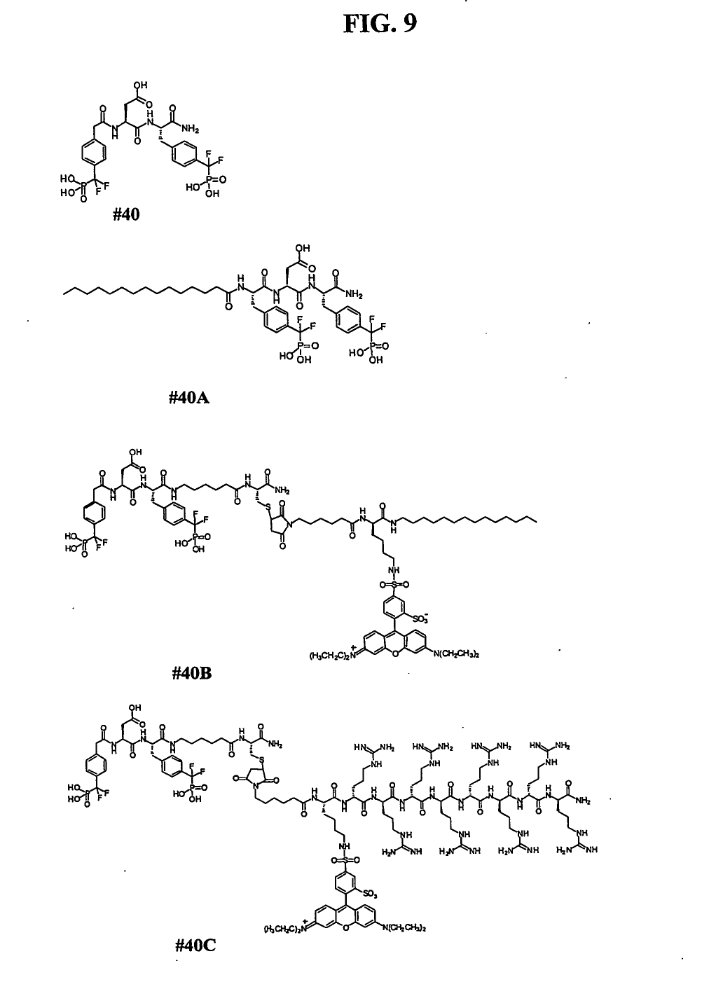

Example 1 describes a highly potent PTP1B inhibitor compound 40. Compound 40 displays a Kᵢ value of 2.4 nM for PTP1B and exhibits several orders of magnitude selectivity in favor of PTP1B against a panel of PTPs. In order to assess the effect of compound 40 in vivo, we have prepared analogs of compound 40, 40A, 40B, and 40C (FIG. 9), in order to promote the membrane permeability of 40. Compounds 40A and 40B involve the conjugation of compound 40 to a fatty acid, while compound 40C involves the attachment of compound 40 to a poly Arg peptide. We found that the stearic acid moiety or the poly Arg peptide do not affect the potency and selectivity of compound 40. Additionally, compounds 40B and 40C include a covalently bound rhodamine molecule to enable the visualization of those compounds, e.g., in cells.

Compound 40A, 40B, and 40C readily penetrate into several cell types, including CHO, COS, HepG2, and L6 cells. FIG. 10 shows rhodamine-fluorescent images (Panel A) which indicate that 40B is cell permeable.

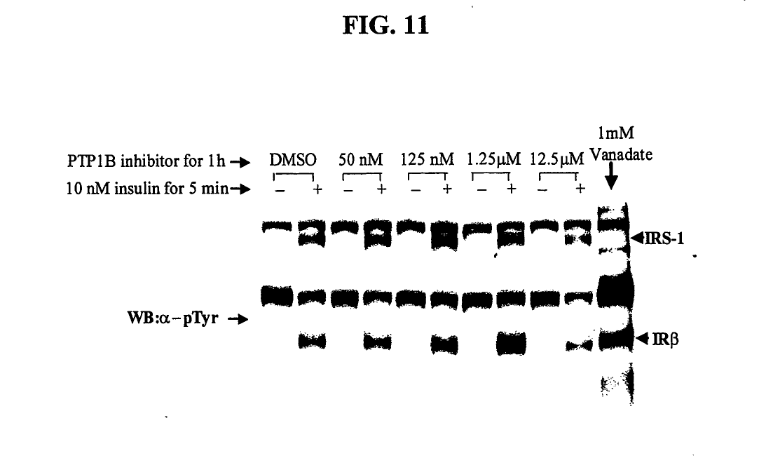

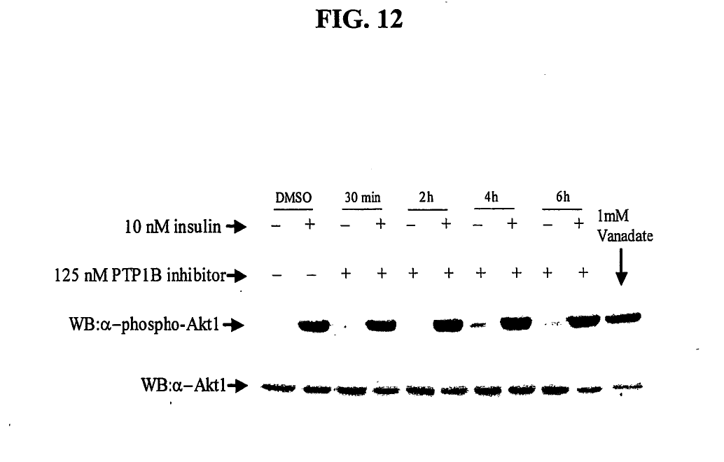

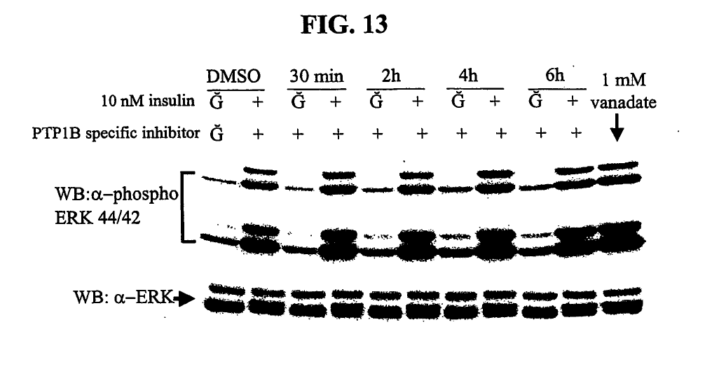

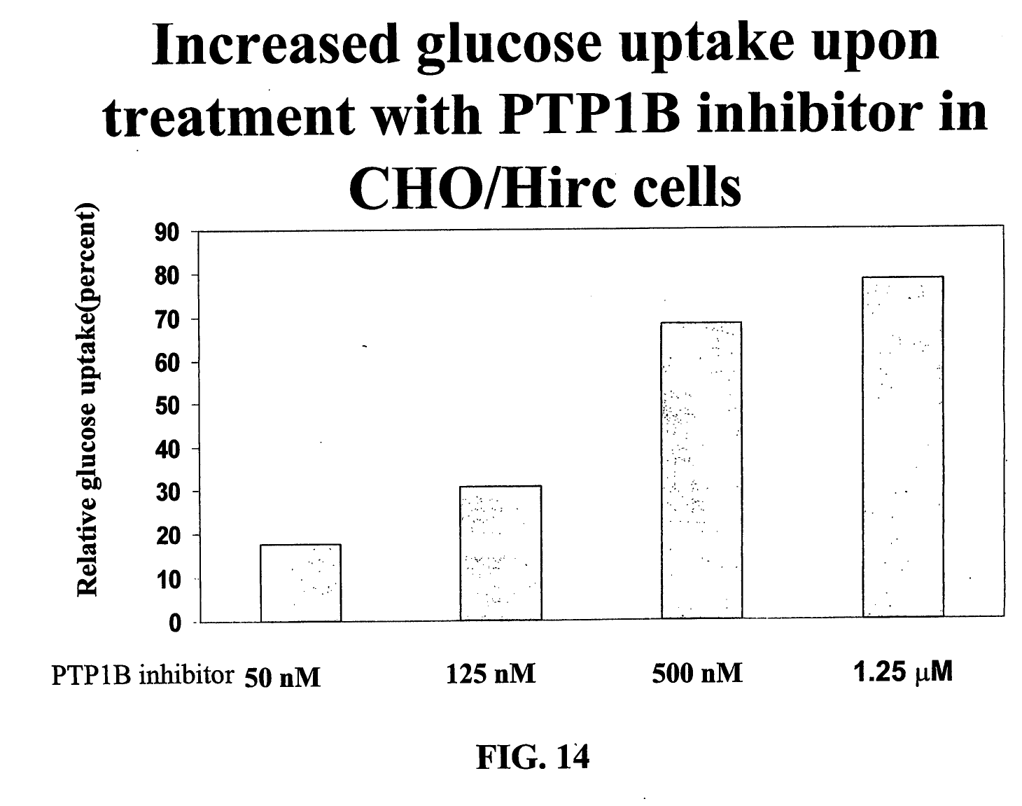

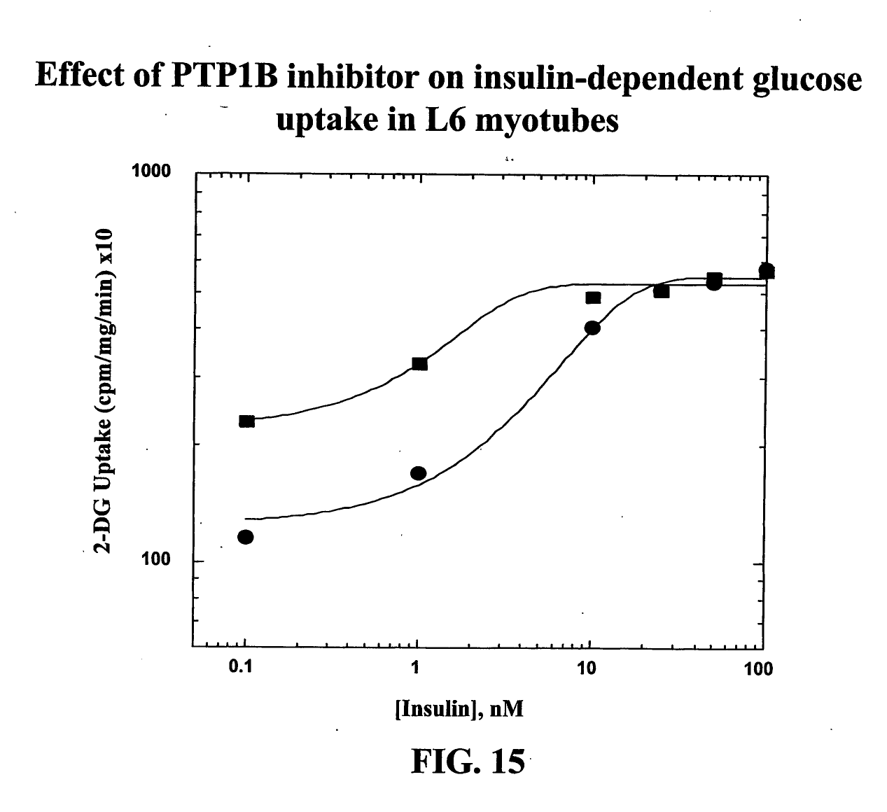

Cellular effects of these compounds were evaluated in several cell lines. Compound 40A enhances tyrosine phosphorylation of both the insulin receptor (IR) β-subunit and the insulin receptor substrate 1 (IRS1) synergistically with insulin in CHO/Hir cells (FIG. 11). In addition, compound 40A further increases insulin-stimulated activation of Akt (FIG. 12) and ERK1/2 kinase activity in the same cell line (FIG. 13). Similar results have been obtained in L6 myotubes. Compound 40A also enhances insulin stimulated glucose uptake in both CHO/Hir and L6 cells (FIGS. 14 and 15). Collectively, these results establish that potent and selective PTP1B inhibitors will augment insulin signaling and may serve as effective therapeutics for the treatment of type II diabetes and obesity.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

References

1. Zhang, Z.-Y. (2001) Curr. Opin. Chem. Biol. 5, 416-423

2. Ahmad, F., Li, P. M., Meyerovitch, J., and Goldstein, B. J. (1995) J. Biol. Chem. 270, 20503-20508

3. Kenner, K. A., Anyanwu, E., Olefsky, J. M., and Kusari, J. (1996) J. Biol. Chem. 271, 19810-19816

4. Bandyopadhyay, D., Kusari, A., Kenner, K. A., Liu, F., Chernoff, J., Gustafson, T. A, and Kusari, J. (1997) J. Biol. Chem. 272, 1639-1645

5. Elchebly, M., Payette, P., Michaliszyn, E., Cromlish, W., Collins, S., Loy, A. L., Normandin, D., Cheng, A., Himms-Hagen, J., Chan, C. C., Ramachandran, C., Gresser, M. J., Tremblay, M. L., and Kennedy, B. P. (1999) Science 283, 1544-1548

6. Klaman, L. D., Boss, O., Peroni, O. D., Kim, J. K., Martino, J. L., Zabolotny, J. M., Moghal, N., Lubidn, M., Kim, Y. B., Sharpe, A. H., Stricker-Krongrad, A., Shulman, G. I., Neel, B. G. and Kahn, B. B. (2000) Mol. Cell. Biol. 20, 5479-5489

7. Jia, Z., Barford, D., Flint, A. J., and Tonks, N. K. (1995) Science 268, 1754-1758

8. Zhang, Z.-Y. (1998) Crit. Rev. Biochem. & Mol. Biol. 33, 1-52

9. Sarmiento, M., Puius, Y. A., Vetter, S. W., Keng, Y. F., Wu, L., Zhao, Y., Lawrence, D. S., Almo, S. C. and Zhang, Z.-Y. (2000) Biochemistry 39, 8171-8179

10. Lawrence, D. S., and Niu, J. (1998) Pharmacol. Ther. 77, 81-114

11. Cohen, P. (1999) Curr. Opin. Chem. Biol. 3, 459-465

12. Zhang, Z.-Y. (1997) Current Topics in Cellular Regulation 35, 21-68

13. Vetter, S. W., Keng, Y. F., Lawrence, D. S. and Zhang, Z.-Y. (2000) J. Biol. Chem. 275, 2265-2268

14. Lee, T. R. and Lawrence, D. S. (1999) J. Med. Chem. 42, 784-787

15. Chen, L., Montserat, J., Lawrence, D. S. and Zhang, Z.-Y. (1996) Biochemistry 35, 9349-9354

16. Launikonis, A, Lay, P. A., Mau, A. W.-H., Sargeson, A. M. and Sasse, W. H. F. (1986) Australian J. Chem. 39, 1053-1062

17. Ganem, B. and Boeckman, R. K. Jr., (1974) Tetrahedron Lett. 917-920

18. Williams, R. M. and Im, M.-N. (1991) J. Amer. Chem. Soc. 113, 9276-9286

19. Yokomatsu, T., Murano, T., Suemune, K. and Shibuya, S. (1997) Tetrahedron 53, 815-822

20. Solas, D., Hale, R. L. and Patel, D. V. (1996) J. Org. Chem. 61, 1537-1539

21. Smyth, M. S.; and Burke T. R., Jr. (1994) Tetrahedron Lett. 35, 551-554

22. Puius, Y. A., Zhao, Y., Sullivan, M., Lawrence, D. S., Almo, S. C. & Zhang, Z.-Y. (1997) Proc. Natl. Acad. Sci. USA 94, 13420-13425

23. Zhang, Z.-Y., Clemens, J. C., Schubert, H. L., Stuckey, J. A., Fischer, M, W. F., Hume, D. M., Saper, M. A. and Dixon, J. E. (1992) J. Biol. Chem. 267, 23759-23766

24. Wu, L. and Zhang, Z.-Y. (1996) Biochemistry 35, 5426-5434

25. Zhang, Z.-Y., Wu, L. and Chen, L. (1995) Biochemistry 34, 16088-16096

26. Zhou, B., Wu, L., Shen, K., Zhang, J., Lawrence, D. S. and Zhang, Z.-Y. (2001) 9J. Biol. Chem. 276, 6506-6515

27. Hao, L., Tiganis, T., Tonks, N. K. and Charbonneau, H. (1997) J. Biol. Chem. 272, 29322-29329

28. Taing, M., Keng, Y. F., Shen, K., Wu, L., Lawrence, D. S., and Zhang, Z.-Y. (1999) Biochemistry 38, 3793-3803

29. Zhang, Z.-Y., Maclean, D., Thieme-Sefler, A. M., McNamara, D., Dobrusin, E. M., Sawyer, T. K. and Dixon, J. E. (1993) Proc. Natl. Acad. Sci. USA 90, 4446-4450

30. Zhang, Z.-Y., Maclean, D., McNamara, D. J., Sawyer, T. K. & Dixon, J. E. (1994) Biochemistry 33, 2285-2290

31. Montserat, J., Chen, L., Lawrence, D. S. and Zhang, Z.-Y. (1996) J. Biol. Chem. 271, 7868-7872

32. Zhang, Z.-Y. and Wu, L. (1997) Biochemistry 36, 1362-1369

33. Zhang, Y.-L., Yao, Z.-J., Sarmiento, M., Wu, L., Burke, T. R. Jr. and Zhang, Z.-Y. (2000) J. Biol. Chem. 275, 34205-34212

34. Burke, T. R., Jr. Smyth, M., Nomizu, M., Otaka, A., and Roller, P. P. (1993) J. Org. Chem. 58, 1336-1340

35. Burke, T. R. Jr., Kole, H. K., and Roller, P. P. (1994) Biochem. Biophys. Res. Commun. 204, 129-134

36. Chen, L., Wu, L., Otaka, A., Smyth, M. S., Roller, P. P., Burke, T. R., den Hertog, J. and Zhang, Z.-Y. (1995) Biochem. Biophys. Res. Commun. 216, 976-984

37. Huyer, G., Kelly, J., Moffat, J., Zamboni, R., Jia, Z., Gresser, M. J. and Rarnachandran, C. (1998) Anal. Biochem. 258, 19-30

38. Desmarais, S., Friesen, R. W., Zamboni, R. and Ramachandran, C. (1999) Biochem. J. 337, 219-223

39. Wrobel, J., Sredy, J., Moxham, C., Dietrich, A., Li, Z., Sawicki, D. R., Seestaller, L., Wu, L., Katz, A., Sullivan, D., Tio, C., and Zhang, Z.-Y. (1999) J. Med. Chem. 42, 3199-3202

40. Iversen, L. F., Andersen, H. S., Branner, S., Mortensen, S. B., Peters, G. H., Norris, K., Olsen, O. H., Jeppesen, C. B., Lundt, B. F., Ripka, W., Moller, K. B., and Moller, N. P. (2000) J. Biol. Chem. 275, 10300-10307

41. Bleasdale, J. E., Ogg, D., Palazuk, B. J., Jacob, C. S., Swanson, M. L., Wang, X. Y., Thompson, D. P., Conradi, R. A., Mathews, W. R., Laborde, A. L., Stuchly, C. W., Heijbel, A., Bergdahl, K., Bannow, C. A., Smith, C. W., Svensson, C., Liljebris, C., Schostarez, H. J., May, P. D., Stevens, F. C., and Larsen, S. D. (2001) Biochemistry 40, 5642-5654

42. Laskowsld, M. and Qasim, M.A. (2000) Biochim. Biophys. Acta 1477, 324-337

43. Burke, T. R. and Zhang, Z. Y. (1998) Biopolymers 47, 225-241

44. PCT patent application WO 00/17211

45. Zabolotny, J. M., Bence-Hanulec, K. K., Stricker-Krongrad, A., Haj, F., Wang, Y., Minokoshi, Y., Kim, Y. B., Elmquist, J. K., Tartaglia, L. A., Kahn, B. B., and Neel, B. G. (2002) Dev. Cell 2, 385-387

46. Cheng, A., Uetani, N., Simoncic, P. D., Chaubey, V. P., Lee-Loy, A., McGlade, C. J., Kennedy, B. P., and Tremblay, M. L. (2002) Dev. Cell 2, 497-503

47. U.S. Pat. No. 6,225,131

48. Shen, K., Keng, Y. F., Wu, L., Guo, X. L., Lawrence, D. S., and Zhang, Z. Y. (2001) J. Biol. Chem. 276, 47311-47319

1. A compound comprising an active site-targeted component, a linker component, and a peripheral site-targeted component, the linker component covalently bound to the active site-targeted component and the peripheral site-targeted component covalently bound to the linker component, wherein the active site-targeted component has a formula as in compound 3 of FIG. 1, and wherein the linker component and the peripheral site-targeted component are each any organic molecule of less than 500 Dalton, the compound further comprising a sulfhydryl group as in compound 3 of FIG. 1.

2 (Cancelled)

3 The compound of claim 1, wherein the linker component consists of carbon, oxygen, nitrogen and/or hydrogen and wherein the peripheral site-targeted component has an aromatic ring and consists of carbon, oxygen, nitrogen, phosphorous, and/or hydrogen.

4 The compound of claim 1, wherein the compound is a ligand of protein tyrosine phosphatase 1B (PTP1B).

5 The compound of any one of claim 50, wherein the linker component is selected from the group consisting of elements 4 through 26 of FIG. 3.

6. The compound of claim 50, wherein the linker component is selected from the group consisting of elements 11, 13, 21, 22, and 24 of FIG. 3.

7 The compound of claim 50, wherein the peripheral site-targeted component is selected from the group consisting of elements A-H of FIG. 2.

8 The compound of claim 50, wherein the peripheral site-targeted component is selected from the group consisting of elements A, B, C, F and H of FIG. 2.

9-18 (cancelled)

19 A ligand of protein tyrosine phosphatase 1B (PTP1B) with an active site-targeted component, a linker component, and a peripheral site-targeted component, as in compound 3 of FIG. 1, wherein the linker component and the peripheral site-targeted component are selected from the group consisting of the following elements of FIGS. 3 and 2, respectively: 4A, 4B, 4C, 4E, 4F, 5A, 5B, 5C, 5F, 6A, 6B, 6E, 6F, 6H, 7A, 7B, 7C, 7E, 7F, 7H, 8A, 8B, 8C, 8F, 8H, 9A, 9B, 9C, 9F, 9H, 10A, 10B, 10C, 10F, 10H, 11A, 11B, 11C, 11D, 11E, 11F, 11G, 11H, 12A, 12B, 12C, 12F, 12G, 12H, 13A, 13B, 13C, 13D, 13E, 13F, 13G, 13H, 14A, 14B, 14C, 15A, 15B, 15C, 15E, 15F, 15H, 16A, 16B, 16C, 16F, 16H, 17A, 17B, 17C, 17E, 17F, 17H, 18A, 18B, 18C, 15E, 18F, 18G, 18H, 19A, 19B, 19C, 19F, 20A, 20B, 20C, 20E, 10F, 20G, 20H, 21A, 21B, 21C, 21D, 21E, 21F, 21G, 21H, 22A, 22B, 22C, 22D, 22E, 22F, 22G, 23H, 24A, 24B, 24C, 24D, 24E, 24F, 24G, 24H, 25F, 26A, 26B, 26C, 26E, 26F, 26G, and 26H; the ligand comprising at least one phosphate group.

20. The ligand of claim 19, wherein the linker component is selected from the group consisting of element 21 and 24 of FIG. 3, and the peripheral site-targeted component is B of FIG. 2.

21. The ligand of claim 19, wherein the linker component is element 21 of FIG. 3 and the peripheral site-targeted component is B of FIG. 2.

22-24 (cancelled)

25 An inhibitor of protein tyrosine phosphatase 1B (PTP1B) with an active site-targeted component, a linker component, and a peripheral site-targeted component, the inhibitor comprising the ligand of claim 19, wherein the at least one phosphate group is substituted with a hydrolytically resistant moiety.

26 The inhibitor of claim 25, consisting of a compound selected from the group consisting of 40, 40A, 40B, and 40C of FIG. 9.

27 A composition comprising the PTP1B inhibitor of claim 25, in a pharmaceutically acceptable excipient.

28-32 (cancelled)

33 A method of inhibiting activity of a PTP1B comprising contacting the PTP1B with the inhibitor of claim 25.

34 The method of claim 33, wherein the PTP1B is in a living cell.

35 The method of claim 34, wherein the cell is in a living mammal.

36 (cancelled)

37 The method of claim 35, wherein the mammal is a human.

38 A method of evaluating whether a compound is a ligand of an enzyme, the method comprising the steps of
(a) combining a known active site ligand of the enzyme with the compound and a mutant of the enzyme, wherein the mutant is capable of binding to a substrate of the enzyme, but not catalyzing the chemical conversion of the substrate; and
(b) determining whether the compound is capable of competing for binding of the known ligand to the mutant of the enzyme, wherein the capacity of the compound to compete for binding indicates that the compound is a ligand for the enzyme.

39-47 (cancelled)

48 A combinatorial library for discovering a ligand of a protein tyrosine phosphatase, comprising more than one form of compound 3 of FIG. 1, wherein X and Y are each independently any organic molecule of less than 500 Dalton.

49 (cancelled)

50 A compound comprising an active site-targeted component, a linker component, and a peripheral site-targeted component, the linker component covalently bound to the active site-targeted component and the peripheral site-targeted component covalently bound to the linker component, wherein the active site-targeted component has a formula as in compound 3 of FIG. 1, and wherein the linker component and the peripheral site-targeted component are each any organic molecule of less than 500 Dalton, wherein the compound is a ligand of protein tyrosine phosphatase 1B (PTP1B).

51 The inhibitor of claim 25, wherein the linker component is selected from the group consisting of element 21 and 24 of FIG. 3, and the peripheral site-targeted component is B of FIG. 2.

52 The inhibitor of claim 25, wherein the linker component is element 21 of FIG. 3 and the peripheral site-targeted component is B of FIG. 2.

53 The inhibitor of claim 25, further comprising a fatty acid moiety.

54 The inhibitor of claim 53, wherein the fatty acid moiety comprises at least 6 carbons.

55 The inhibitor of claim 53, wherein the fatty acid moiety comprises at least 10 carbons.

56 The inhibitor of claim 53, wherein the fatty acid moiety comprises 15 carbons.

57 The inhibitor of claim 25, further comprising a polyarginine moiety.

58 The inhibitor of claim 57, wherein the polyarginine moiety comprises at least 4 arginines.

59 The inhibitor of claim 57,wherein the polyarginine moiety comprises 8 arginines.

60 The inhibitor of claim 25, further comprising a detectable moiety.

61 The inhibitor of claim 60, wherein the detectable moiety is a fluorescent moiety.

62 The inhibitor of claim 60, wherein the detectable moiety is a rhodamine.

63 The inhibitor of claim 25, wherein the hydrolytically resistant moiety is selected from the group consisting of phosphonodifluoromethyl and difluorophosphonate.

64 The inhibitor of claim 25, wherein the hydrolytically resistant moiety is difluorophosphonate.
